SEQUENCE LISTING (1 sequence)

SEQ ID NO: 1 - protein, 8 aa, synthetic construct
SIINFEKL

(19) United States
(12) Patent Application Publication
(10) Pub. No.: US 2026/0069709 A1
(43) Pub. Date: Mar. 12, 2026

(54) EXTRACELLULAR VESICLE HYDROGELS AND USE THEREOF

(71) Applicant: The Board of Trustees of the University of Illinois, Urbana, IL (US)

(72) Inventors: Hua Wang, Champaign, IL (US); Rimsha Bhatta, Urbana, IL (US)

(73) Assignee: The Board of Trustees of the University of Illinois, Urbana, IL (US)

(21) Appl. No.: 19/323,127

(22) Filed: Sep. 9, 2025

Related U.S. Application Data
(60) Provisional application No. 63/692,788, filed on Sep. 10, 2024.

(51) Int. Cl. A61K 47/69; A61K 39/00; A61K 39/395; A61K 47/60; A61P 35/00

(52) U.S. Cl. CPC A61K 47/6901; A61K 39/3955; A61K 47/60; A61P 35/00; A61K 2039/505

(57) ABSTRACT

Hydrogels including a plurality of extracellular vesicles covalently linked to a polymer, wherein the covalent link includes a glycan moiety, are provided. Methods of making and using the hydrogels, for example, for treating or inhibiting cancer, are also provided.

### CROSS REFERENCE TO RELATED APPLICATIONS

This application claims priority to U.S. Provisional Application No. 63/692,788, filed Sep. 10, 2024, which is incorporated by reference in its entirety.

### ACKNOWLEDGMENT OF GOVERNMENT SUPPORT

This invention was made with government support under R01 CA274738 and R21 CA270872 awarded by the National Institutes of Health and under 2143673 awarded by the National Science Foundation. The government has certain rights in the invention.

### FIELD

This disclosure relates to extracellular vesicle hydrogels and their use, particularly for treating or inhibiting cancer in a subject.

### INCORPORATION OF ELECTRONIC SEQUENCE LISTING

The Sequence Listing is submitted as an XML file in the form of the file named “7950-112430-02_Sequence_Listing” (1930 bytes), which was created on Aug. 28, 2025, which is incorporated by reference herein.

### BACKGROUND

Extracellular vesicles (EVs) are small (micro- or nano-sized) lipid vesicles secreted by nearly all types of cells and which comprise a variety of molecules including lipids, proteins, RNAs, and other metabolites from the parent cells. Hence, they play a critical role in cellular communication and have raised interest in exploring their therapeutic applications. For example, EVs derived from mesenchymal stem cells are being actively explored as therapeutics for tissue injuries including myocardial infarction and muscle injury. Tumor cell-derived EVs contain tumor-associated antigens which can be processed and presented by antigen presenting cells in the body, for subsequent elicitation of tumor-specific T cell response. These EV-based therapies hold promise for eventual clinical translation, but are currently limited by modest efficacy. In current practice, EVs are often administered in a solution form into the tissue of interest or systemically into the bloodstream. The poor tissue retention of EVs, despite the administration of a high number of EVs or multiple doses, often dampens the overall efficacy.

### SUMMARY

There remains a need for EV compositions with improved tissue retention and efficacy. Provided herein are hydrogels that include a plurality of extracellular vesicles covalently linked to a polymer, wherein the covalent link includes a glycan moiety. In some aspects, the plurality of extracellular vesicles are covalently linked to the polymer by click chemistry (such as azide-alkyne click chemistry, tetrazine-norbornene click chemistry, tetrazine-cyclooctene click chemistry, or maleimide-thiol click chemistry).

In some aspects, the plurality of EVs are from one or more tumor cells (such as tumor cells from a subject with cancer). In some examples, the EVs include exosomes.

In some aspects the polymer includes polyethylene glycol, poly lactic-co-glycolic acid, or polycaprolactone. In some examples, the polymer is modified with a linker (for example, dibenzocyclooctyne). The disclosed hydrogels may also include an adjuvant, such as CpG.

Also provided are methods of producing the EV hydrogels. In some aspects, the methods include providing a plurality of azido-labeled extracellular vesicles, wherein the azido-label is an azido-labeled glycan moiety (for example, tetra-acetylated N-azidoacetyl-D-mannosamine (Ac₄ManNAz), tetra-acetylated N-azidoacetyl-D-galactosamine (Ac₄GalNAz), or tetra-acetylated N-azidoacetyl-D-glucosamine (Ac₄GlcNAz)); and covalently coupling the plurality of azido-labeled extracellular vesicles to a polymer functionalized with a linker capable of covalently binding the azido-labeled extracellular vesicles. In some aspects, the plurality of azido-labeled extracellular vesicles are produced by culturing a plurality of cells (such as plurality of tumor cells) in the presence of an azido-labeled glycan moiety. In one example, the functionalized polymer is polyethylene glycol functionalized with dibenzocyclooctyne. In some aspects, covalently coupling the plurality of azido-labeled extracellular vesicles to the functionalized polymer includes mixing the azido-labeled extracellular vesicles and the functionalized polymer and incubating at about 37° C. to form the hydrogel.

Methods of treating a subject with a tumor are also provided. In some aspects, the methods include administering to the subject an effective amount of a disclosed EV hydrogel, wherein the plurality of extracellular vesicles included in the hydrogel are derived from one or more tumor cells (such as one or more tumor cells from the subject). In some examples, the methods include injecting the hydrogel or a hydrogel precursor subcutaneously, at or near the tumor location, or intraperitoneally. The methods may further include administering to the subject one or more immune checkpoint inhibitors (such as an anti-PD-1 antibody, an anti-PD-L1 antibody, and/or an anti-CTLA-4 antibody).

The foregoing and other feature of the disclosure will become more apparent from the following detailed description, which proceeds with reference to the accompanying figures.

### BRIEF DESCRIPTION OF THE DRAWINGS

FIGS. 1A and 1B illustrate the formation of hydrogels using extracellular vesicles (EVs) and their use in therapeutic cancer vaccines. FIG. 1A is a schematic illustration of metabolic tagging of EVs and subsequent formation of EV hydrogels via click chemistry. FIG. 1B schematically illustrates EV hydrogels for cancer vaccines or immunotherapies. Injectable EV hydrogels can persist for weeks in vivo, allowing for continuous attraction of dendritic cells (DCs) for the processing and presentation of EV-encased tumor antigens. Tumor antigen-presenting DCs can then migrate to lymph nodes to prime tumor-specific CD8⁺ T cells for eventual tumor killing.

FIGS. 2A-2J illustrate the synthesis and characterization of EV hydrogels. FIG. 2A is a schematic illustration of labeling of cancer cells with azido group and subsequent secretion of azido-labeled EVs after 3 days of incubation. FIG. 2B shows Cy5 fluorescence intensity of E.G7-OVA EVs that were incubated with DBCO-Cy5 for 30 min. Also shown are the average diameter (FIG. 2C), relative concentration (FIG. 2D), and TEM images (FIG. 2E) of azido-labeled EVs and control EVs derived from E.G7-OVA cells. FIG. 2F is a schematic showing the formation of EV hydrogels from azido-labeled EVs and DBCO-PEG via click chemistry. FIG. 2G shows images of the mixture of DBCO-PEG and azido-labeled EVs or control EVs at 37° C. FIG. 2H shows representative plots and FIG. 2I shows average storage moduli (G′) and loss Moduli (G″) of formed EV hydrogels. FIG. 2J is a SEM image of EV hydrogel. All the numerical data are presented as mean±SD (0.01<*P≤0.05; **P≤0.01; ***P≤0.001; ****P≤0.0001).

FIGS. 3A-3H illustrate EV hydrogels with tunable gelling temperature, storage moduli, and stress relaxation half-lives. EV gels were formed by mixing 8-arm DBCO-PEG and different concentrations of azido-labeled EVs. An EV concentration of 7×10⁹/mL, 1.75×10⁹/mL, 1.17×10⁹/mL, 0.875×10⁹/mL, and 0.70×10⁹/mL was used for EV gel-1, EV gel-2, EV gel-3, EV gel-4, and EV gel-5, respectively. Shown are the changes of storage moduli (G′) and loss moduli (G″) of EV gel-1 (FIG. 3A), EV-gel 2 (FIG. 3B), and EV-gel 3 (FIG. 3C) at constant strain (γ=0.5%) during heating from 20° C. to 80° C., measured at a heating speed of 5° C./min. Changes of G′ and G″ over time at 37° C. are shown for EV gel-1 (FIG. 3D), EV gel-2 (FIG. 3E), and EV gel-3 (FIG. 3F). FIG. 3G shows representative stress relaxation curves and FIG. 3H shows stress relaxation half-lives of EV gel 1-5.

FIGS. 4A-4I show that EV hydrogels gradually release EVs for subsequent processing of EV-encased antigens by DCs. FIG. 4A shows porosity of EV gel-1, EV gel-2, EV gel-3 and EV gel-4. FIG. 4B shows change in the volume of gels over time in vitro. FIG. 4C shows percentage of volume loss over time in vitro for EV gel 1-4. FIGS. 4D-4F: DCs were incubated with EV gel, the mixture of EV gel and CpG (EV gel+CpG), CpG-conjugated EV gel (EV/CpG gel), or PBS for 16 h. Shown are percentage of CD86⁺MHCII⁺ DCs (FIG. 4D), mean CD86 FI of DCs (FIG. 4E), and mean MHCII FI of DCs (FIG. 4F). FIGS. 4G-4H: DCs pretreated with EV gel, EV gel+CpG, EV/CpG gel, OVA+CpG, or PBS were cocultured with CFSE-stained OT-1 cell for three days. Shown are representative CFSE histograms of OT-1 cells (FIG. 4G) and proliferation index of OT-1 cells in different groups (FIG. 4H). All the numerical data are presented as mean±SD (0.01<*P≤0.05; **P≤0.01; ***P<0.001; ****P<0.0001). FIG. 4I shows photos of EV gel 1, EV gel 2, EV gel 3, and EV gel 4 immersed in PBS for different times. An EV concentration of 7×10⁹/mL, 1.75×10⁹/mL, 1.17×10⁹/mL, and 0.875×10⁹/mL was used for EV gel-1, EV gel-2, EV gel-3, and EV gel-4, respectively.

FIGS. 5A-5J show EV gel stability in vivo and their recruitment and modulation of DCs in situ. FIGS. 5A-5C: Mice were subcutaneously injected with the mixture of Cy5-conjugated EVs and DBCO-PEG, which formed a gel rapidly after injection. FIG. 5A shows IVIS images of mice at different times post injection. FIG. 5B shows the change of EV gel volume over time. FIG. 5C shows change of Cy5 fluorescence intensity of EV gels over time. FIGS. 5D-5J: Mice were subcutaneously injected with the mixture of azido-labeled EVs (with or without conjugation of DBCO-CpG) and DBCO-PEG, which formed a gel rapidly after injection. Gels were harvested for immune analysis after three days. Shown are percentage of DCs (FIG. 5D), macrophages (FIG. 5E), neutrophils (FIG. 5F), and T cells (FIG. 5G) among the recruited cells in EV gels. FIG. 5G shows number of neutrophils, macrophages, DCs, and T cells in EV gels. FIG. 5I shows CD86 MFI of DCs in EV gels. FIG. 5J shows MHCII MFI of DCs in EV gels.

FIGS. 6A-6I show that CpG-conjugated EV gels enhanced CTL response and antitumor efficacy. FIG. 6A is a schematic timeframe of the vaccination study. EV gel, EV gel+CpG, EV/CpG gel, OVA+CpG, or PBS were subcutaneously injected into C57BL/6 mice on day 0. E.G7-OVA tumor cells were inoculated on day 15. Shown are the percentages of MHCI-SIINFEKL (SEQ ID NO: 1) tetramer⁺ CD8⁺ T cells (FIGS. 6B, 6D, and 6F) and IFN-r⁺ CD8⁺ T cells (FIGS. 6C, 6E, and 6G) in PBMCs on day 6, 9, and 14, respectively. FIG. 6H shows average E.G7-OVA tumor volume of each group over the course of the prophylactic tumor study. FIG. 6I shows Kaplan-Meier plots for all groups. All the numerical data are presented as mean±SD except for (h) where data are presented as mean±SEM.

FIGS. 7A-7H show CpG-conjugated EV gel synergy with α-PD-1 for tumor treatment. FIG. 7A is a schematic diagram of timeframe of the therapeutic tumor study against E.G7-OVA tumors. E.G7-OVA tumor was inoculated on day 0. EV/CpG gel or EV gel was subcutaneously injected on day 9. α-PD-1 was i.p. administered on days 13, 16 and 19. FIG. 7B shows individual tumor curves for each group. FIG. 7C shows average E.G7-OVA tumor volume of each group over the course of the therapeutic tumor study. FIG. 7D shows Kaplan-Meier plots were used for all groups. FIGS. 7E-7H: C57BL/6 mice were subcutaneously injected with EV/CpG gel, EV gel, the mixture of uncrosslinked EV and CpG, or PBS on day 0. Shown are the anti-OVA IgG titers in the serum of mice on day 5 (FIG. 7E), day 7 (FIG. 7F), day 12 (FIG. 7G), and day 18 (FIG. 7H), respectively. Anti-OVA titers were measured via ELISA assay. Data in FIG. 7C are presented as mean±SEM and data in FIGS. 7E-7H are presented as mean±SD.

FIGS. 8A and 8B show E.G7-OVA cells incubated with different concentrations of Ac₄ManNAz for three days, followed by incubation with DBCO-Cy5 for 30 min to detect cell-surface azido groups. FIG. 8A shows percentages of Cy5+ cells. FIG. 8B shows mean Cy5 fluorescence intensity of cells.

FIG. 9 shows an estimation of the number of azido groups per EV. EVs were collected from Ac₄ManAz-treated E.G7-OVA cells. The fluorescence intensity of DBCO-Cy5 being conjugated to azido-labeled EVs was measured, which was subtracted with the background Cy5 signal from unlabeled EVs. In 100 μl of EV-Cy5 solution, the measured Cy5 FI: 52,420; calculated Cy5 amount: 2.936×10⁻¹³ moles; Calculated number of Cy5 molecules: 1.77×10¹¹; measured number of EVs (via NTA): 3.8×10⁷; calculated number of Cy5 per EV: 4658, estimated number of azide per EV>4658.

FIGS. 10A-10D show EVs and azido-labeled EV characteristics. FIG. 10A shows images of EVs and azido-labeled EVs, as obtained from NTA. FIG. 10B shows size and size distribution of EVs and azido-labeled EVs, as determined by NTA. FIGS. 10C-10D: EVs were isolated from three different batches of E.G7-OVA cells on day 5.

FIG. 10C shows number of E.G7-OVA cells from 3 different batches. FIG. 10D shows concentration of EVs from 3 different batches.

FIG. 11 shows proteomic analysis of EV and EV-N₃ derived from E.G7-OVA cells. Two different batches of EVs were analyzed.

FIG. 12 shows the ¹H NMR spectrum of 8-arm DBCO-PEG in MeOD.

FIGS. 13A-13C show strain sweep tests of storage (G′) and loss (G″) moduli of EV gel-1 (FIG. 13A), EV gel-2 (FIG. 13B), and EV gel-3 (FIG. 13C), respectively, under the frequency of 1 Hz.

FIG. 14 shows quantification of EV-N₃ before and after mixing with DBCO-PEG to form EV gel-1.

FIGS. 15A-15C show stability of EV gels at 37° C. in 10% FBS in vitro. FIG. 15A shows photos of EV gel 1, EV gel 2, EV gel 3, and EV gel 4 immersed in cell culture media (10% FBS) at 37° C. for different times. An EV concentration of 7×10⁹/mL, 1.75×10⁹/mL, 1.17×10⁹/mL, and 0.875×10⁹/mL was used for fabricating EV gel-1, EV gel-2, EV gel-3, and EV gel-4, respectively. Scale bar: 3 mm. FIG. 15B shows change in the volume of gels at 37° C. over time in vitro. FIG. 15C shows percentage of volume loss for EV gels at 37° C. over time in vitro.

FIG. 16 shows Western blot analysis of OVA in E.G7-OVA derived EVs. Pure OVA protein and EVs collected from B16F10 cells were used as controls.

FIGS. 17A and 17B show data from EV gels incubated at 37° C. for up to 2 weeks. An aliquot of the medium was collected on day 1, 3, 5, 9, and 14, respectively for ELISA assay to quantify the released OVA from EV gels. FIG. 17A shows accumulated release of OVA from EV gels over time. FIG. 17B shows percentages of released OVA over time.

FIGS. 18A-18B show viability of DCs cultured with EV gels for different times (FIG. 18A) and viability of DCs in regular culture media (FIG. 18B).

FIGS. 19A and 19B show percentages of CD86⁺ DCs after incubation with different groups for 16 h (FIG. 19A) or percentages of CD86⁺ macrophages after incubation with different groups for 16 h (FIG. 19B). DCs or macrophages were added to the top of EV gels (CpG-conjugated EV gel, mixture of EV gel and CpG, or EV gel) or incubated with the solution of non-crosslinked EVs (mixture of EV and DBCO-PEG or mixture of EV, DBCO-PEG, and CpG).

FIGS. 20A-20B show Cy5 fluorescence from Cy5-conjugated EVs. FIG. 20A shows confocal images of BMDCs after 1 h incubation with Cy5-conjugated EVs. Cell nuclei were stained with DAPI (blue) and cell membranes were stained with Cell Mask Stain (green), respectively. Scale bar: 20 μm. FIG. 20B shows Cy5 fluorescence intensity of DCs after incubation with Cy5-conjugated EVs or control EVs for 6 hours as determined via flow cytometry.

FIGS. 21A-21C show calcein AM-stained dendritic cells were loaded into EV gels (4 mm diameter×2 mm height) and incubated at 37° C. Scale bar: 150 μm. FIG. 21A shows fluorescence images of DCs on EV gels at different times. FIG. 21B shows quantification of DCs in EV gels over time. FIG. 21C shows images of EV gels with or without loading of DCs over time. Scale bar: 2 mm.

FIG. 22 shows IVIS images of mice at different times post injection. Mice were subcutaneously injected with the mixture of Cy5-conjugated EVs (7×10⁹/mL) and DBCO-PEG (27 mg/mL), which formed a gel rapidly after injection.

FIGS. 23A-23C shows mice were subcutaneously injected with the mixture of azido-labeled EVs (with or without conjugation of DBCO-CpG) and DBCO-PEG, which formed a gel rapidly after injection. Gels were harvested for immune analysis after three days. FIG. 23A shows gating strategy for flow cytometry analysis. FIG. 23B shows percentages of CD45+ cells in gels. FIG. 23C shows percentages of CD11b⁺ cells among CD45+ cells in gels.

FIGS. 24A-24G show images of EV/CpG gel and EV gel at 7 days after subcutaneous injection (FIG. 24A). Percentages of DCs (FIG. 24B), neutrophils (FIG. 24C), and T cells (FIG. 24D) in the fibrous capsule surrounding the gel at 3 days post gel injection. Percentages of DCs (FIG. 24E), neutrophils (FIG. 24F), and T cells (FIG. 24G) in the fibrous capsule surrounding the gel at 7 days post gel injection.

FIGS. 25A-25B show EV gel, EV gel+CpG, EV/CpG gel, OVA+CpG or PBS were subcutaneously injected into C57BL/6 mice on day 0. FIG. 25A shows representative FACS plots of tetramer⁺ CD8⁺ T cells in PBMC on days 6, 9, and 14, respectively. FIG. 25B shows representative FACS plots of IFN-γ⁺ CD8⁺ cells in PBMC on days 6, 9, and 14, respectively, following ex vivo stimulation with SIINFEKL (SEQ ID NO: 1) peptide.

FIG. 26 shows Western blot analysis of Tsg101 and CD63 in E.G7-OVA derived EVs.

FIGS. 27A-27B show release profile of OVA from the OVA-encapsulating alginate hydrogel. Aliquots of the medium were collected at different times, for ELISA assay to quantify the released OVA from gels. Shown are accumulated amount of released OVA (FIG. 27A) and percentage of OVA released from alginate gels (FIG. 27B) at different times.

FIGS. 28A-28B show quantitation of number of EVs released from EV hydrogel using Nanoparticle Tracking Analysis. FIG. 28A shows number of EVs released from EV hydrogel and FIG. 28B shows total percentage of EV released from EV hydrogel.

FIGS. 29A-29B show MHCII MFI of DCs (FIG. 29A) and percentages of MHCI-SIINFEKL (SEQ ID NO: 1)⁺ DCs (FIG. 29B) after incubation with different groups for 16 h. DCs were added to the top of EV gels (EV gel, CpG-conjugated EV gel, or CpG-encapsulating EV gel) or incubated with the solution of non-crosslinked EVs (without or with CpG). All the numerical data are presented as mean±SD.

FIGS. 30A-30C show percentages of CD163⁺ macrophages (FIG. 30A), CD206⁺ macrophages (FIG. 30B), and CD86⁺ macrophages (FIG. 30C) after incubation with different groups for 16 h. Macrophages were added to the top of EV gels (CpG-conjugated EV gel, mixture of EV gel and CpG, or EV gel) or incubated with the solution of non-crosslinked EVs (mixture of EV and DBCO-PEG or mixture of EV, DBCO-PEG, and CpG).

FIG. 31 shows IVIS images of mice at different times post-subcutaneous injection of Cy5-congjugated EV alone.

FIGS. 32A-32D show results from mice subcutaneously injected with EV/CpG gel or EV gel or PBS. The capsule tissue or skin tissue surrounding the gels was harvested for immune analysis after 3 or 7 days. FIG. 32A shows percentages of CD4⁺ T cells in the surrounding skin tissue on day 3. FIG. 32B shows percentages of CD8⁺ T cells in the surrounding skin tissue on day 3. FIG. 32C shows percentages of CD4⁺ T cells in the surrounding skin tissue on day 7. FIG. 32D shows percentages of CD8⁺ T cells in the surrounding skin tissue on day 7. All the numerical data are presented as mean±SD.

FIG. 33 shows cytokine expression profiles at the injection site at 3 days post injection of CpG-conjugated EV gel, EV gel, the non-crosslinked mixture of EV, DBCO-PEG, and CpG, or PBS (untreated).

FIGS. 34A-3L show EV gel treatment does not induce significant changes in blood cell populations. CpG-conjugated EV gels or EV gels were subcutaneously injected into the flank of C57BL/6 mice on day 0. Blood was drawn on days 3, 6, and 9, respectively for the analysis of blood cells. FIG. 34A shows relative concentration of RBCs on day 3. FIG. 34AB shows percentages of monocytes on day 3. FIG. 34C shows percentages of granulocytes on day 3. FIG. 34D shows percentages of lymphocytes on day 3. FIG. 34E shows relative concentration of RBCs on day 6. FIG. 34F shows percentages of monocytes on day 6. FIG. 34G shows percentages of granulocytes on day 6. FIG. 34H shows percentages of lymphocytes on day 6. FIG. 34I shows relative concentration of RBCs on day 9. FIG. 34J shows percentages of monocytes on day 9. FIG. 34K shows percentages of granulocytes on day 9. FIG. 34L shows percentages of lymphocytes on day 9. All the numerical data are presented as mean±SD.

FIG. 35 shows representative images of H&E stained tissues after treatment of C57BL/6 mice with EV gel, EV gel+CpG, EV/CpG gel, OVA+CpG, EV gel or PBS. Scale bar: 50 μm.

FIGS. 36A-36M show that CpG-conjugated EV gel reprograms the tumor microenvironment. E.G7-OVA tumor was inoculated to C57BL/6 mice on day 0, followed by the subcutaneous injection of CpG-conjugated EV gel (EV/CpG gel), CpG-encapsulating EV gel, EV gel, the solution of OVA and CpG, the solution of EV alone, the solution of EV, DBCO-PEG, and CpG, or PBS on day 12. Tumors were harvested for analysis on day 15. FIG. 36A shows the timeframe of immune analysis study. FIG. 36B shows % CD3⁺ cells among CD45⁺ population. FIG. 36C shows % CD8⁺ cells among CD45⁺ population. FIG. 36D shows % CD4⁺ cells among CD45⁺ population. FIG. 36E shows % CD69⁺ cells among CD8⁺ population. FIG. 36F shows % FoxP3⁺ cells among CD45⁺CD3⁺CD4⁺ population. FIG. 36G shows the number ratio of CD8 to Tregs. FIG. 36H shows % CD11c⁺ DCs among CD11b⁺CD45⁺ population. FIG. 36I shows % CD86⁺ cells among CD11c⁺ DCs. FIG. 36J shows % CD86⁺ cells among CD11b⁺F4/80⁺ macrophages. FIG. 36K shows % CTLA-4⁺ cells among CD8⁺ T cells. FIG. 36L shows % PD-1⁺ cells among CD8⁺ T cells. FIG. 36M shows % LAG-3⁺ cells among CD8⁺ T cells. All the numerical data are presented as mean±SD.

FIGS. 37A-37G show E.G7-OVA tumor inoculated to C57BL/6 mice on day 0, followed by the subcutaneous injection of CpG-conjugated EV gel (EV/CpG gel), CpG-encapsulating EV gel, EV gel, the solution of OVA and CpG, EV alone, the non-crosslinked solution of EV, DBCO-PEG and CpG, or PBS on day 12. Gels and surrounding tissues were harvested for analysis on day 15. FIG. 37A is the timeframe of immune cell analysis study. FIG. 37B shows % CD8⁺ cells among CD45⁺ population. FIG. 37C shows % CD4⁺ cells among CD45⁺ population. FIG. 37D shows % CD69⁺ cells among CD8⁺ population. FIG. 37E shows % CD11c⁺ DCs among CD11b⁺CD45⁺ population. FIG. 37F shows % CD86⁺ cells among CD11c+DCs. FIG. 37G shows % F4/80⁺ cells among CD45⁺CD11b⁺ populations. All the numerical data are presented as mean±SD.

FIGS. 38A-38B show size distribution (FIG. 38A) and zeta potential (FIG. 38B) of EVs from E.G7-OVA cells, as measured by dynamic light scattering (DLS).

### SEQUENCE LISTING

Any nucleic acid and amino acid sequences listed herein or the accompanying Sequence Listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NO: 1 is an ovalbumin peptide: SIINFEKL

### DETAILED DESCRIPTION

Nearly all types of mammalian cells can secrete EVs to transport cellular contents and mediate intercellular communication. Hence, EVs have long been an attractive candidate for diagnostics and therapeutics. As disclosed herein, EVs, via facile metabolic tagging, can be covalently crosslinked into a gel network with tunable stiffness and relaxation half-lives. The EVs serve as the multifunctional backbone to form the crosslinked gel network. Injectable EV hydrogels are stable for weeks and exhibit excellent retention in vivo. In the context of cancer vaccine, it is demonstrated that tumor EV hydrogels, fabricated using the approach described herein, can serve as a long-term depot of EVs, facilitate the processing and presentation of EV-encased tumor antigens by dendritic cells, and result in persistent tumor-specific CD8⁺ T cell response and therapeutic efficacy.

### I. Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various aspects of the disclosure, the following explanations of specific terms are provided.

Biocompatible: The property of a biomaterial or device having the ability to perform its desired function (for example, with respect to a medical therapy), without eliciting any undesirable local or systemic effects (such as undesirable immune responses) in a subject. A biocompatible material or device ideally also generates a beneficial effect or cellular or tissue response. In some examples, biocompatible refers to a material or device that is enzymatically or chemically degraded in vivo into simpler chemical species (“biodegradable”). A biocompatible material, device, or system includes synthetic or natural material used to function in close contact with living tissue.

Contacting: Placement in direct physical association; includes both in solid and liquid form.

Click chemistry: A category of chemical reactions typically used to join a molecule of interest with a specific biomolecule. Click reactions occur in a single vessel, are not disturbed by water, generate minimal and non-toxic byproducts, and are characterized by a high thermodynamic driving force that drives it quickly and irreversibly to high yield of a single reaction product, with high reaction specificity.

Click chemistry reactions include [3+2] cycloadditions (e.g., Huisgen 1,3-dipolar cycloaddition), thiol-ene reaction, Diels-Alder reaction, [4+1] cycloaddition between isonitrile and tetrazine, azide-alkyne reactions, tetrazine-norbornene reactions, tetrazine-cyclooctene reactions, and maleimide-thiol reactions.

Covalently coupled, conjugated, or linked: Coupling a first unit to a second unit. This includes, but is not limited to, covalently bonding one molecule to another molecule (for example, directly or via a linker molecule), noncovalently bonding one molecule to another (e.g. electrostatically bonding), non-covalently bonding one molecule to another molecule by hydrogen bonding, non-covalently bonding one molecule to another molecule by van der Waals forces, and any and all combinations of such couplings. In one example, conjugating includes covalent bond linkage of a glycoprotein or glycolipid (such as a glycoprotein or glycolipid including a non-naturally occurring sugar moiety on an exosome) to a polymer. The covalent bond linkage can be direct or indirect, e.g., linked though a spacer molecule or other linker molecule.

Extracellular vesicle (EV): EVs are cell-derived, membrane-surrounded vesicles secreted by nearly all types of cells. They carry a variety of molecules from the parent cell, including, but not limited to lipids, proteins, nucleic acids (such as DNA or RNA), and/or other metabolites. In one non-limiting example, an EV is derived from a tumor cell and includes one or more tumor-associated antigens.

Exosome: Exosomes are a class of cell-derived extracellular vesicles of endosomal origin and can be about 30-150 nm in diameter. Enveloped by a lipid bilayer, exosomes are released into the extracellular environment and contain components derived from the original cell, such as, but not limited to, proteins, lipids, RNA (such as DNA or RNA (for example, mRNA, and/or miRNA)), and/or DNA. Exosomes are formed through the fusion and exocytosis of multivesicular bodies into the extracellular space. Multivesicular bodies are organelles in the endocytic pathway that function as intermediates between early and late endosomes. A function of multivesicular bodies is to separate components that will be recycled elsewhere from those that will be degraded by lysosomes. The vesicles that accumulate within multivesicular bodies are categorized as intraluminal vesicles while inside the cytoplasm and exosomes when released from the cell. Intraluminal vesicles are thus essentially exosome precursors, and form by budding into the lumen of the multivesicular body. In some examples, exosomes are derived from tumor cells, immune cells, or stem cells, and can be isolated from a supernatant of a cell culture of a population of such cells using methods described herein.

Hydrogel: A network of polymer chains that are hydrophilic, sometimes found as a colloidal gel in which water is the dispersion medium. Hydrogels are highly absorbent natural or synthetic polymeric networks. Hydrogels also possess a degree of flexibility similar to natural tissue. In some examples, the hydrogels disclosed herein comprise a plurality of EVs covalently linked to a polymer.

Inhibiting or treating a disease: Inhibiting the full development of a disease or condition, for example, in a subject who is at risk for a disease, such as a subject with cancer. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Isolated: An “isolated” or “purified” biological component (such as a cell, nucleic acid, peptide, protein, or EV) has been substantially separated, produced apart from, or purified away from other components (for example, other biological components in the cell or environment in which the component naturally occurs). Cells, nucleic acids, peptides and proteins, or EVs that have been “isolated” or “purified” thus include cells, nucleic acids, proteins, and EVs purified by methods known to one of ordinary skill in the art.

The term “isolated” or “purified” does not require absolute purity; rather, it is intended as a relative term. Thus, for example, an isolated biological component is one in which the biological component is more enriched than the biological component is in its starting environment, such as within a cell, organism, sample, or production vessel (for example, a cell culture system). Preferably, a preparation is purified such that the biological component represents at least 50%, such as at least 70%, at least 80%, at least 90%, at least 95%, or greater, of the total biological component content of the preparation.

Pharmaceutically acceptable carrier: Remington: The Science and Practice of Pharmacy, Adejare (Ed.), Academic Press, London, United Kingdom, 23^{rd} Edition (2021), describes compositions and formulations suitable for pharmaceutical delivery of one or more therapeutic compounds or molecules, such as one or more disclosed EV or EV hydrogel preparations, and/or additional pharmaceutical agents.

Purified: The term purified does not require absolute purity; rather, it is intended as a relative term. Thus, for example, a purified EV preparation is one in which the exosome is more enriched than in its original environment. In one aspect, a preparation is purified such that a component (such as purified EVs) represents at least 50% of the total content of the preparation.

Sample or biological sample: Biological material obtained from a subject, which can include cells, proteins, and/or nucleic acid molecules. Biological samples include all clinical samples useful for detection, analysis, or treatment of a disease, such as cancer, in subjects. Appropriate samples include any conventional biological samples, including clinical samples obtained from a human or veterinary subject. Exemplary samples include, without limitation, cancer or tumor samples (such as from surgery, tissue biopsy, tissue sections, or autopsy), cells, cell lysates, blood smears, cytocentrifuge preparations, cytology smears, bodily fluids (e.g., blood, plasma, serum, saliva, sputum, urine, bronchoalveolar lavage, semen, cerebrospinal fluid (CSF), etc.), or fine-needle aspirates. Samples may be used directly from a subject, or may be processed before further use (such as concentrated, diluted, purified, or expanded or maintained in culture). In a particular example, a sample or biological sample is obtained from a subject having, suspected of having, or at risk of having cancer, for example, a tumor cell or plurality of tumor cells.

Subject: As used herein, the term “subject” refers to a mammal and includes, without limitation, humans, domestic animals (e.g., dogs or cats), farm animals (e.g., cows, horses, or pigs), and laboratory animals (e.g., mice, rats, hamsters, guinea pigs, pigs, rabbits, dogs, or monkeys). In some aspects the subject has a disease or disorder, such as cancer.

Therapeutically effective amount: The amount of an active ingredient that is sufficient to effect treatment when administered to a mammal in need of such treatment, such as treatment of a cancer. The therapeutically effective amount will vary depending upon the subject and disease condition being treated, the weight and age of the subject, the severity of the disease condition, the manner of administration and the like, which can readily be determined by a prescribing physician.

Tumor, neoplasia, malignancy or cancer: A neoplasm is an abnormal growth of tissue or cells which results from excessive cell division. Neoplastic growth can produce a tumor. The amount of a tumor in an individual is the “tumor burden,” which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, non-cancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

### II. EV Hydrogels

Provided herein are hydrogels that include a plurality of extracellular vesicles covalently linked to a polymer, referred to in some examples as “EV hydrogels.” As disclosed herein, the EVs serve as the multifunctional backbone to form the crosslinked gel network.

In some aspects, the disclosed hydrogels include a plurality of extracellular vesicles covalently linked to a polymer, wherein the covalent link includes a glycan moiety. In some examples, the glycan moiety includes an azido-acetylated sugar moiety that can be incorporated into a glycoprotein or glycolipid, such as acetylated N-azidoacetyl-D-mannosamine (Ac₄ManNAz), tetra-acetylated N-azidoacetyl-D-galactosamine (Ac₄GalNAz), tetra-acetylated N-azidoacetyl-D-glucosamine (Ac₄GlcNAz), N-azidoacetyl-D-mannosamine (ManNAz), N-azidoacetyl-D-galactosamine (GalNAz), N-azidoacetyl-D-glucosamine (GlcNAz), or 9-azido-9-deoxy-N-acetylneuraminic acid (9AzNeu5Ac).

In certain examples, the plurality of extracellular vesicles are covalently linked to the polymer by click chemistry, such as azide-alkyne click chemistry, tetrazine-norbornene click chemistry, tetrazine-cyclooctene click chemistry, or maleimide-thiol click chemistry. In one specific example, the plurality of EVs are covalently linked to the polymer by azide-alkyne click chemistry (such as utilizing azido-labeled EVs covalently linked to DBCO-modified polymer).

In some aspects, the EVs are exosomes (also referred to as “small EVs”), microvesicles, apoptotic bodies or apoptotic vesicles, autophagic EV, stressed or damaged EVs, matrix vesicles, large oncosomes, migrasomes, ectosomes, exomeres, supermeres, membrane particles, or a mixture of two or more thereof. See, e.g., Sheta et al., Biology (Basel) 12(1):110, 2023. In particular examples, the EVs are derived from a tumor cell from a subject. In other examples, the EVs are derived from a stem cell (including, but not limited to a mesenchymal stem cell), or a pathogenic cell (including, but not limited to E. coli).

In some aspects, the metabolically labeled EVs are crosslinked to a modified or functionalized polymer to form the EV hydrogel. In some examples, the polymer is a biocompatible polymer. Examples of polymers suitable for hydrogel formation include naturally occurring polymers (such as hyaluronic acid, chitosan, heparin, alginate, gelatin, or fibrin) or synthetic polymers or copolymers (such as polyethylene glycol, poly lactic-co-glycolic acid, polycaprolactone, polyvinyl alcohol, poly acrylic acid, polylactic acid, polyglycolic acid, or polyacrylamide). In some examples, the polymer is a linear polymer. In other examples, the polymer is a multi-arm (such as 2-arm, 3-arm, 4-arm, 5-arm, 6-arm, 7-arm, 8-arm, 9-arm, 10-arm, 12-arm, 16-arm, or more) polymer. In one example, the polymer is functionalized multi-arm PEG, such as functionalized 8-arm PEG. In one non-limiting example, the functionalized polymer is 8-arm PEG modified with DBCO.

In some examples, the EV hydrogel includes about 1×10⁷ EV/ml to about 1×10¹⁰ EV/ml (such as about 1×10⁷ EV/ml to about 1×10⁸ EV/ml, about 1×10⁸ EV/ml to about 1×10⁹ EV/ml, or about 1×10⁹ EV/ml to about 1×10¹⁰ EV/ml). In some examples, the EV hydrogel includes about 7×10⁹ EV/ml, about 5×10⁹ EV/ml, about 2×10⁹ EV/ml, about 1.5×10⁹ EV/ml, about 1×10⁹ EV/ml, or about 0.5×10⁹ EV/ml.

The EV hydrogels are prepared as a liquid mixture and form a hydrogel when exposed to a suitable gelation temperature (e.g., about 37° C.), as discussed in Section III. In some examples, the liquid including the metabolically labeled EVs and functionalized polymer is a buffer or other suitable pharmaceutically acceptable carrier (including but not limited to, phosphate buffered saline or Hank's balanced salt solution).

In some aspects, the EV hydrogel further includes an adjuvant, such as CpG, lipopolysaccharide, polyI:C, alum, imiquimod, or motolimod. In one specific example, the adjuvant is CpG. In some examples, the EV hydrogel includes about 0.01 μg to about 100 μg CpG per gel, such as about 0.01 μg to about 0.1 μg per gel, about 0.05 μg to about 0.5 μg per gel, about 0.12 μg to about 0.75 μg per gel, about 0.1 μg to about 1 μg per gel, about 0.5 μg to about 5 μg per gel, about 1 μg to about 10 μg per gel, about 2.5 μg to about 15 μg per gel, about 10 μg to about 25 μg per gel, about 20 μg to about 50 μg per gel, or about 50 μg to about 100 μg per gel. In other examples, the amount of CpG in the EV hydrogel corresponds about 0.0005 mg/kg to about 5 mg/kg CpG, for example, about 0.0005 mg/kg to about 0.001 mg/kg, about 0.001 mg/kg to about 0.05 mg/kg, about 0.05 mg/kg to about 0.1 mg/kg, about 0.1 mg/kg to about 1 mg/kg, or about 1 mg/kg to about 5 mg/kg. In some examples, the adjuvant is incorporated into the hydrogel by mixing the metabolically labeled EVs and the functionalized polymer with the adjuvant to form a crosslinked EV/adjuvant hydrogel. In other examples, the adjuvant is encapsulated into the EV hydrogel.

### III. Methods of Producing EV Hydrogels

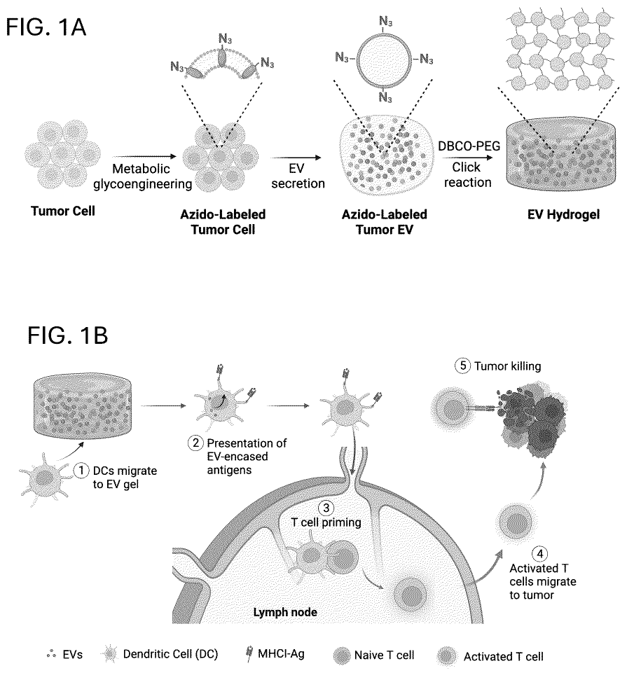

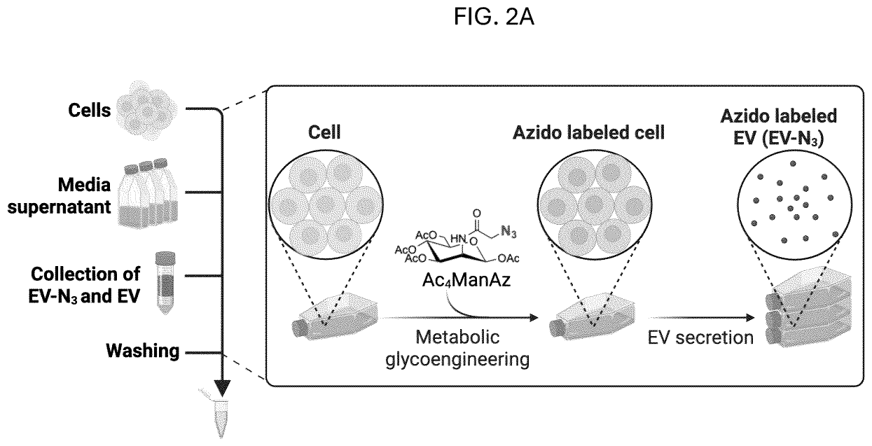

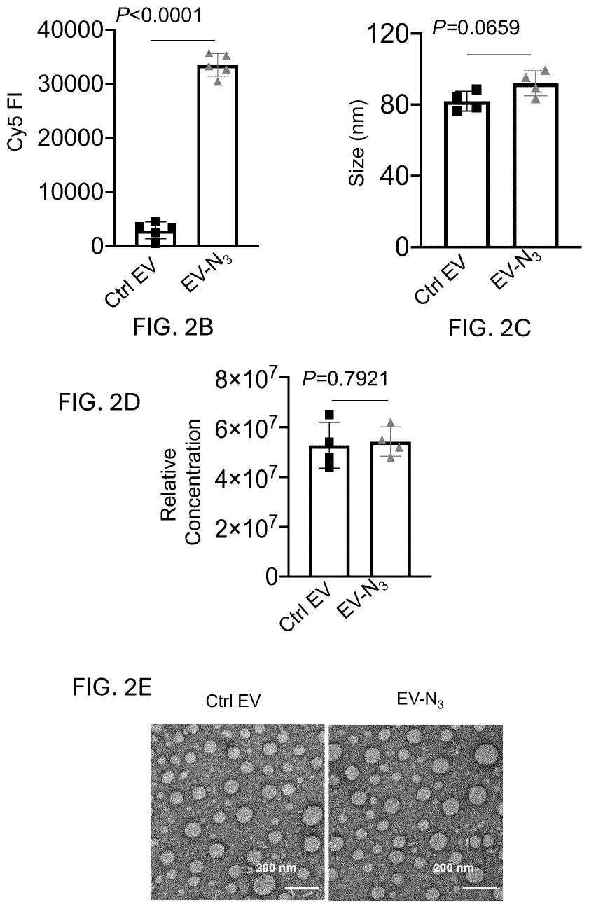

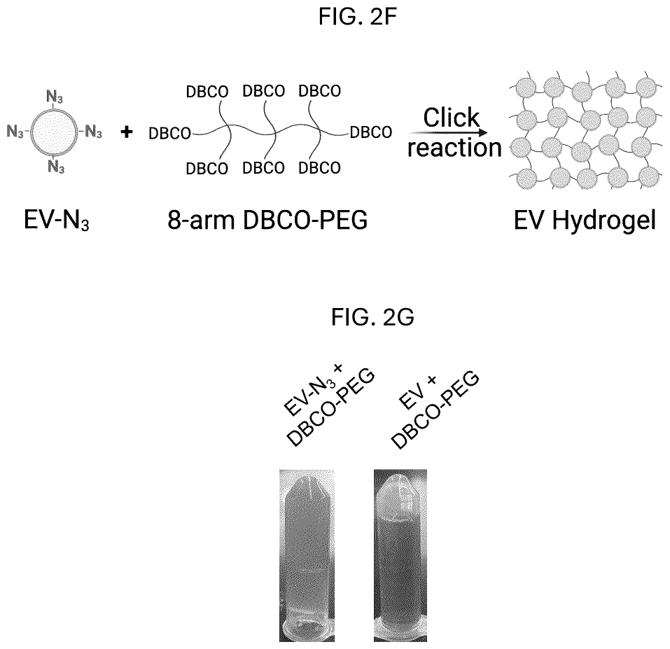

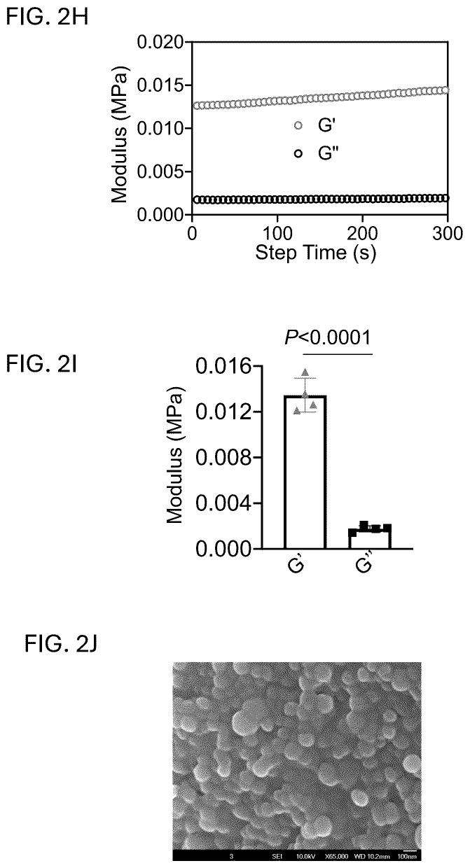

Also provided are methods of preparing the EV hydrogels disclosed herein. Metabolic glycan labeling of cells provides a facile approach to generating chemically tagged EVs. Once internalized by cells, unnatural sugars bearing clickable chemical tags can undergo metabolic glycoengineering processes, conjugate to proteins and lipids, and become expressed on the cell membrane in the form of glycoproteins and glycolipids (Wang and Mooney, Nature Chemistry 12:1102-1114, 2020). Since EVs inherit a portion of the cell membrane, EVs secreted by the labeled cells will also carry the chemical tags. In one non-limiting example, azido-tagged EVs can react with dibenzocyclooctyne (DBCO)-functionalized 8-arm polyethylene glycol (PEG) via efficient click chemistry to yield a fully crosslinked gel network. Exemplary methods for producing EV hydrogels are illustrated in FIGS. 1A and 2A. Particular EVs (such as EVs from tumor cells labeled with Ac₄ManNAz) are illustrated herein; however, these methods can be utilized with any type of cell and with other non-naturally occurring sugars.

In some aspects, the metabolic labeling process utilizes non-naturally occurring sugars that can be incorporated into glycoproteins or glycolipids and can be used to covalently couple the glycoprotein to a molecule of interest via a chemical tag on the sugar, for example using click-chemistry methods. In some aspects, the methods utilize an azido-acetylated sugar moiety that can be incorporated into a glycoprotein or glycolipid, such as acetylated N-azidoacetyl-D-mannosamine (Ac₄ManNAz), tetra-acetylated N-azidoacetyl-D-galactosamine (Ac₄GalNAz), tetra-acetylated N-azidoacetyl-D-glucosamine (Ac₄GlcNAz), N-azidoacetyl-D-mannosamine (ManNAz), N-azidoacetyl-D-galactosamine (GalNAz), N-azidoacetyl-D-glucosamine (GlcNAz), or 9-azido-9-deoxy-N-acetylneuraminic acid (9AzNeu5Ac). In one aspect, Ac₄ManNAz is taken up by cells, and is hydrolyzed by esterases, followed by phosphorylation and ring-opening isomerization and conversion to sialic acid by attack by phosphoenolpyruvic acid. The sialic acid is conjugated to a protein and expressed on the surface of an exosome in the form of a glycoprotein. In other aspects, the azido-acetylated sugar moiety may also include a trigger-responsive moiety that is cleaved by a trigger (such as a trigger that is enhanced or increased in cancer cells) and a linker, such as a self-immolative linker. Exemplary trigger-responsive moieties are described in International Patent Application Publication No. WO 2017/062800, which is incorporated herein by reference in its entirety.

Thus, in some aspects, the methods include culturing cells of interest (such as a cancer cell, an immune cell, or a stem cell) in vitro in the presence of an azido-labeled sugar moiety (such as about 0.1-200 μM, for example, about 0.1-5 μM, about 1-10 μM, about 5-15 M, about 10-25 μM, about 20-40 μM, about 30-50 M, about 50-75 μM, about 60-100 μM, about 80-125 M, about 100-150 M, or about 150-200 μM). In one example, the cells are cultured with 50 UM azido-labeled sugar (such as 50 μM Ac₄ManNAz). In other aspects, the cells are cultured in the presence of a non-naturally occurring sugar including N-modified mannosamine, 6-modified fucose, N-modified galactosamine, or N-modified glucosamine which are modified with a chemical tag selected from azide, diazoalkane, cyclopropene, isonitrile, alkene, diazirine, DBCO, alkyne, or ketone (e.g., as described in Wang and Mooney, Nature Chemistry, 12:1102-1114, 2020, incorporated herein by reference in its entirety). In some examples, the cells are cultured for a period of time prior to metabolic labeling, for example, to allow the cells to attach to a culture vessel surface and/or to proliferate to provide a sufficient number of cells for labeling. In some examples, the cells are obtained or isolated from a subject, such as a subject with a disease or disorder (for example, a subject with cancer). In some examples, the cells are tumor cells, such as tumor cells isolated or obtained from a subject with cancer.

After a sufficient period of time in culture for incorporation of the non-naturally occurring sugar into cell surface proteins or lipids (for example, at least 30 minutes, at least 1 hour, at least 2 hours, at least 4 hours, at least 8 hours, at least 12 hours, at least 24 hours, at least 36 hours, at least 48 hours, at least 72 hours, at least 96 hours, or more, for example, at least 1, 2, 3, 4, 5, 6, 7, or more days), EVs are collected from the cell culture (e.g., from the cell culture medium). In some examples, the non-naturally occurring sugar is added to the culture medium one time, while in other examples, fresh medium including the non-naturally occurring sugar is added one or more times during the culture. The isolated EVs include one or more labeled surface glycoproteins or glycolipids.

EVs may be isolated from a supernatant of a culture of cells using various methods known in the art. Such methods include, but are not limited to, centrifugation (such as ultracentrifugation, such as serial ultracentrifugation), charge neutralization-based precipitation, gel-filtration/size-exclusion chromatography (GF/SEC), immunoaffinity techniques (such as affinity purification using immunogenic beads), purification with magnetic beads, ultrafiltration (such as stirred ultrafiltration), double filtration using microfluidic devices, nanoplasmon-enhanced scattering, and lab-on-a-chip devices (such as acoustic nanofiltration, immunoaffinity, filtration, trapping on nanowires, viscoelastic flow sorting, and/or lateral displacement).

In some aspects, EVs are isolated from the supernatant of a culture of cells that have been metabolically labeled with an azido-labeled sugar moiety using centrifugation, such as ultracentrifugation or serial ultracentrifugation. In some examples, the supernatant of a labeled culture of cells is centrifuged in successive rounds with increasing centrifugation forces and durations to remove cells, cellular debris, and/or macromolecular proteins, followed by ultracentrifugation to obtain isolated labeled EVs. In some examples, serial ultracentrifugation is used to isolate EVs from a portion of, substantially all, or all other components of a cell culture supernatant, such as a supernatant from a culture of azido-labeled cells.

In some aspects, the isolated labeled EVs may be further purified, for example, by size exclusion chromatography, for example to remove free proteins in the medium. In one example, the EVs are further purified using a qEV size exclusion column. In another example, EVs and free proteins may be separated using differential centrifugation.

Isolated EVs can be quantified using a variety of methods. Such methods include, but are not limited to nanoparticle tracking analysis, flow cytometry, tunable resistive pulse sensing, electron microscopy, mass spectrometry (for example, to quantify EVs based on the level of one or more proteins known to be present in the EVs), dynamic light scattering, and microfluidic devices. For example, EVs can be quantified using commercially available kits, such as the NanoSight NS300 Exosome Quantitation Kit (System Biosciences, Palo Alto, CA, USA).

The one or more labeled EV surface proteins or lipids are then covalently coupled to a functionalized polymer to form the EV hydrogel. In particular aspects, the functionalized polymer is covalently coupled to an azido-labeled EV surface protein or lipid utilizing click chemistry, such as azide-alkyne click chemistry. Depending on the chemical tag included in the non-naturally occurring sugar, one of ordinary skill in the art can select appropriate click chemistry methods, including azido-alkyne click chemistry, tetrazine-norbornene click chemistry, tetrazine-cyclooctene click chemistry, or maleimide-thiol click chemistry. In one example, the polymer is modified with DBCO, which can be coupled to an azido-labeled EV. One of ordinary skill in the art can select appropriate modifications based on the chemical tag in the non-naturally occurring sugar used to metabolically label the EVs.

In some aspects, the metabolically labeled EVs are mixed with the functionalized polymer at incubated at a temperature resulting in formation of a hydrogel. In some examples, the metabolically labeled EVs and functionalized polymer are incubated at about 36-40° C. (such as about 36° C., about 37° C., about 38° C., about 39° C., or about 40° C.) to form the EV hydrogel. In some examples, the mixture is liquid at room temperature and forms the EV hydrogel when administered to (e.g., injected) a subject, resulting in exposing the mixture to a temperature of about 37° C. (e.g., about mammalian (such as human) body temperature). In some examples, varying the amount of metabolically labeled EVs with respect to the amount of functionalized polymer can be used to “tune” the gelation temperature of the hydrogel. In one non-limiting example, an amount of about 7×10⁹ EVs per 20 mg functionalized polymer resulted in a gelation temperature of 39.4° C., which also was demonstrated to form a hydrogel when injected into a subject (e.g. at about 36-40° C., such as about 37° C.).

In some aspects, the methods are used to prepare a cancer vaccine or immunotherapy agent. Cancer or tumor cells from a subject are cultured in vitro in the presence of a non-naturally occurring sugar moiety with a chemical tag, such as Ac₄ManNAz. In this case, the cancer cell produces azido-labeled proteins or lipids, for example, azido-labeled glycoproteins or glycolipids, which in some examples are incorporated into EVs and released by the cancer cell. The EVs are collected or isolated and are formed into a hydrogel as described above.

In other aspects, the EVs are from a stem cell (such as an embryonic stem cell, an induced pluripotent stem cell a mesenchymal stem cell, a neural crest cell, or a hematopoietic stem cell). In other aspects, the EVs are from a pathogenic cell, such as a bacterial cell (for example, E. coli).

### IV. Methods of Use

In some aspects, methods of treating or inhibiting cancer with the EV hydrogels described herein are provided. The EV hydrogels are administered to a subject to treat or inhibit the cancer.

In some aspects, subject being treated has cancer, such as a solid tumor. Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma, and retinoblastoma). In some non-limiting examples, the cancer is lymphoma, melanoma, glioblastoma, or breast cancer (such as triple-negative breast cancer).

In other aspects, subject being treated has a hematological malignancy. Examples of hematological malignancies include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), T-cell large granular lymphocyte leukemia, polycythemia vera, lymphoma, diffuse large B-cell lymphoma, Hodgkin's lymphoma, non-Hodgkin's lymphoma (indolent and high grade forms), mantle cell lymphoma, follicular cell lymphoma, multiple myeloma, Waldenstrom's macroglobulinemia, heavy chain disease, myelodysplastic syndrome, hairy cell leukemia and myelodysplasia.

In some aspects, the EV hydrogel is prepared from tumor cells (such as tumor cells from the subject), for example, as described in Section III. In some examples, a therapeutically effective amount of the hydrogel can be administered to a subject with cancer. In some examples, about 10-200 μl of the hydrogel (such as a liquid hydrogel precursor) is administered to the subject, for example, about 10-30 μl, about 25-50 μl, about 40-80 μl, about 75-100 μl, about 90-125 μl, about 110-130 μl, about 125-150 μl, about 140-180 μl, or about 175-200 μl. A skilled clinician can determine the amount of hydrogel to be administered, for example, based on pre-clinical studies, animal model studies, the condition being treated, the subject's overall condition, and other factors.

In other aspects, the EV hydrogel is prepared from stem cells (such as mesenchymal stem cells) or from pathogenic cells (such as E. coli), for example s described in Section III. In some examples, a therapeutically effective amount of the hydrogel can be administered to a subject in need of, such as a subject who has had a myocardial infarction or who has had an allotransplant, or for the treatment or inhibition of a pathogenic disease, such as bacterial infection. In some examples, a therapeutically effective amount of the hydrogel can be administered to the subject. In some examples, about 10-200 μl of the hydrogel (such as a liquid hydrogel precursor) is administered to the subject, for example, about 10-30 μl, about 25-50 μl, about 40-80 μl, about 75-100 μl, about 90-125 μl, about 110-130 μl, about 125-150 μl, about 140-180 μl, or about 175-200 μl. A skilled clinician can determine the amount of hydrogel to be administered, for example, based on pre-clinical studies, animal model studies, the condition being treated, the subject's overall condition, and other factors.

The hydrogel is administered to the subject by any appropriate route of administration. In some examples, the hydrogel or a hydrogel precursor (such as a mixture of metabolically labeled EVs and functionalized polymer that is liquid at room temperature and forms the hydrogel upon exposure of temperatures of about 37° C.) is administered at or close to a tumor (e.g., local administration) in the subject. In other examples, the hydrogel or hydrogel precursor is administered subcutaneously or intraperitoneally. In one specific example, the hydrogel or hydrogel precursor is administered to the subject subcutaneously via needle injection. One of skill in the art can determine appropriate routes of administration.

In some examples, the subject is administered a single dose of the hydrogel. In other examples, multiple doses (such as 2 or more doses) of the hydrogel can be administered to the subject. For example, the hydrogel can be administered daily, every other day, every three days, twice weekly, weekly, every other week, monthly, or less frequently. A skilled clinician can select an appropriate administration schedule based on the subject, the condition being treated, and other factors.

In some aspects, the hydrogel is administered in combination with (for example, sequentially or simultaneously with) one or more additional treatments for the disease or disorder of the subject. In some non-limiting examples, the subject has cancer, and the hydrogel is administered in combination with one or more additional cancer therapies, such as one or more immune checkpoint inhibitors. In some examples, the immune checkpoint inhibitor is anti-PD-1 (such as nivolumab or pembrolizumab), anti-PD-L1 (such as atezolizumab, avelumab, or durvalumab), and/or anti-CTLA-4 (such as ipilimumab). A skilled clinician can select additional appropriate therapies and administration schedules based on the subject, the disease or disorder being treated, and other factors.

### EXAMPLES

The following examples are provided to illustrate particular features of certain aspects of the disclosure, but the scope of the claims should not be limited to those features exemplified.

### Example 1

### Materials and Methods

This example describes the materials and methods utilized for the experiments described in Examples 2-10.

Materials and Instrumentation. DBCO-Cy5, DBCO-Cy3 and other chemical reagents were purchased from Sigma Aldrich unless otherwise noted (St. Louis, MO, USA). 8-Arm PEG-amine was purchased from JenKem Technology USA (Plano, TX, USA). DBCO-NHS was purchased from Click Chemistry Tools (Scottsdale, AZ, USA). Primary antibodies such as fluorophore conjugated anti-CD45, anti-CD11b, anti-CD11c, anti-CD86, anti-MHCII, anti-CD3, anti-CD4, anti-CD8, anti-Gr-1, and anti-F4/80 were obtained from Thermo Fisher Scientific (Waltham, MA, USA). Ovalbumin Polyclonal Antibody HRP, goat anti-mouse-IgG secondary antibody, and HRP-conjugated streptavidin were obtained from Thermo Fisher Scientific (Waltham, MA, USA). Ovalbumin (OVA) protein was obtained from InvivoGen (San Diego, CA, USA). qEV isolation columns were purchased from IZON Science (Christchurch, New Zealand). Mouse CD3⁺ T cell isolation kit, Dynabeads, and LS separation columns were purchased from Miltenyi Biotec (Bergisch Gladbach, Germany). FACS analyses were performed on an Attune NxT. A BioTek plate reader (Winooski, Vermont, U.S.) was used to measure the fluorescence intensity of solutions and cells. A Shimadzu LC40 ultra high-performance liquid chromatography/mass spectrometer was used to characterize small molecules and proteins. Proton nuclear magnetic resonance spectra were collected on the Varian U500 or VXR500 (500 MHz) spectrometer. Scanning electron microscopic (SEM) images were taken with a Hitachi S-4800 High Resolution Scanning Electron Microscope. Mechanical tests were performed on an AR-G2 rheometer (TA Instruments, New Castle, DE, USA). The size and size distribution of exosomes were measured on a Nanoparticle Tracking Analysis (NTA) system (Malvern, United Kingdom).

Cell lines and animals. The E.G7-OVA cell line was purchased from American Type Culture Collection (Manassas, VA, USA). Cells were cultured in DMEM containing 10% FBS, 100 units/mL Penicillin G, 100 μg/mL streptomycin, and 50 μg/mL G418 at 37° C. in 5% CO₂ humidified air. Female C57BL/6 and OT-I mice were purchased from the Jackson Laboratory (Bar Harbor, ME, USA). All procedures involving animals were done in compliance with National Institutes of Health and Institutional guidelines with approval from the Institutional Animal Care and Use Committee at the University of Illinois at Urbana-Champaign.

Synthesis of Ac₄ManNAz. Ac₄ManNAz was synthesized as follows. In brief, D-Mannosamine hydrochloride and triethylamine in methanol were added N-(2-azidoacetyl) succinimide. After stirring at room temperature for 24 h, the solvent was removed, and the residue was re-dissolved in pyridine. Acetic anhydride was then added and stirred at room temperature for another 24 h. The crude product was purified by silica gel column chromatography using ethyl acetate/hexane (1/1, v/v) as the eluent to yield a white solid (1/1 a/B isomers). ¹H NMR (CDCl₃, 500 MHz): δ (ppm) 6.66&6.60 (d, J=9.0 Hz, 1H, C(O)NHCH), 6.04&6.04 (d, 1H, J=1.9 Hz, NHCHCHO), 5.32-5.35&5.04-5.07 (dd, J=10.2, 4.2 Hz, 1H, CH₂CHCHCH), 5.22&5.16 (t, J=9.9 Hz, 1H, CH₂CHCHCH), 4.60-4.63&4.71-4.74 (m, 1H, NHCHCHO), 4.10-4.27 (m, 2H, CH₂CHCHCH), 4.07 (m, 2H, C(O)CH₂N₃), 3.80-4.04 (m, 1H, CH₂CHCHCH), 2.00-2.18 (s, 12H, CH₃C(O)). ¹³C NMR (CDCl₃, 500 MHZ): δ (ppm) 170.7, 170.4, 170.3, 169.8, 168.6, 168.3, 167.5, 166.9, 91.5, 90.5, 73.6, 71.7, 70.5, 69.1, 65.3, 65.1, 62.0, 61.9, 52.8, 52.6, 49.9, 49.5, 21.1, 21.0, 21.0, 20.9, 20.9, 20.9, 20.8. ESI MS (m/z): calculated for C₁₆H₂₂N₄O₁₀Na [M+Na]⁺ 453.1, found 453.1.

Flow cytometry analysis of cells. Cells were incubated with Ac₄ManNAz (50 μM) or PBS for 3 days. After washing, cells were incubated with DBCO-Cy5 (10 μM) for 30 min and were further washed prior to analysis on a flow cytometer.

EV isolation and characterization. E.G7-OVA cells (1×10⁶) were cultured in the presence of Ac₄ManNAz (50 μM) or PBS for 3 days. The cell culture media was collected on days 4 or 5, after which it was subjected to initial centrifugation at 350 g for 5 minutes to remove cell debris and larger particles. The supernatant was then collected and further purified using ultrafiltration. An Amicon filter with a 100 kDa molecular weight cutoff was used for the ultrafiltration to remove all molecules with molecular weights lower than 100 kDa. The ultrafiltration was conducted using centrifuge 5910 Ri (Eppendorf) at 4,347 g for 40 minutes. At least 5 ultrafiltration washing steps (using PBS, 4,347 g, 40 minutes) were performed to purify EVs. The size and concentration of EVs were determined on the NTA instrument. For the NTA, we used the Malvern NanoSight NS300 instrument. The measurement volume was 500 μL. The length of the video was 10 secs each and the number of videos taken was 5 per sample. The instrument settings has been listed below: [Capture Settings: Camera Type: sCMOS; Laser Type: Blue488; Camera Level: Manual settings used; Slider Shutter: 1300; Slider Gain: 512; FPS 25.0; Number of Frames: 1498; Temperature: 22.5-22.5° C.; Viscosity: (Water) 0.941-0.942 cP; Dilution factor: Dilution not recorded; Syringe Pump Speed: 100. Analysis Settings: Detect Threshold: 17; Blur Size: Auto; Max Jump Distance: Auto: 2.0-14.8 pix]. To validate the presence of azido groups on the surface of EVs, EVs derived from Ac₄ManNAz-treated E.G7-OVA cells were incubated with DBCO-Cy5 (10 μM) for 30 min, washed with PBS via ultrafiltration (100 kDa Amicon filter, 4,347 g, 40 minutes), and the Cy5 fluorescence intensity was measured on a plate reader.

Uptake of EVs by DCs. DC 2.4 cells were seeded onto coverslips in a 6-well plate at a density of 6×10⁶ cells per well and allowed to attach for 48 h. Cy5-conjugated EVs (synthesized via the conjugation of EV-N₃ and DBCO-Cy5) or control EVs were incubated with DC 2.4 cells at 37° C. for 1 h. After washing with PBS, cells were fixed with 4% paraformaldehyde solution, followed by the staining of cell nuclei and membrane with DAPI and cell mask stain, respectively. The coverslips were mounted onto microscopic slides with Prolong-gold and imaged under a confocal laser scanning microscope.

TEM imaging of EVs. Isolated EVs were added onto formvar/carbon-coated TEM grids (Ted Pella, Redding, CA), allowed to dry, negatively stained with 2% aqueous uranyl acetate, and imaged with a JEOL 2100 TEM at 200 kV.

Fabrication of EV gels. E.G7-OVA cells (1×10⁶) were metabolically labeled with Ac₄ManNAz for three days, for subsequent secretion of azido-labeled EVs. Purified azido-labeled EVs (7×10⁹/mL, 1.75×10⁹/mL, 1.17×10⁹/mL, 0.875×10⁹/mL, or 0.7×10⁹/mL final concentration) were then mixed with 8-arm DBCO-PEG (20 mg/mL final concentration) for 3 min at 37° C. to form EV gels. For some experiments, CpG adjuvant was incorporated into the EV gel by either conjugating azido-labeled EVs with) DBCO-modified CpG and further mixing with 8-arm DBCO-PEG to form the crosslinked EV/CpG Gel, or physically encapsulating CpG into EV gels.

Porosity measurement. EV gels were first placed in DI water for 1-2 h. EV gels were then placed on a Kimwipe paper to dry one side of the gel, so that the water inside the pores can come out through capillary action. The weight of gels before and after the wicking assay was measured, and the porosity of the gel was calculated as (Wᵢ-W_{f})/Wᵢ.

Gel volume analysis. EV gels were placed in the medium for 2 weeks at ambient temperature. The length and width of gels were monitored at different times, and the volume of gels was calculated via V=(W²×L)/2.

EV measurement before and after EV gel formation. Initially, EVs were suspended in PBS, and the concentration of EVs in PBS was measured using the Nanoparticle Tracking Analysis (NTA) instrument. EVs in PBS were then mixed with DBCO-PEG to form the gel. After the EV gel was formed, the remaining PBS solution that did not incorporate into the gel was collected, and the gel was washed with fresh PBS. The combined PBS solutions were analyzed by the NTA to measure the concentration of EVs. The total number of EVs was calculated by multiplying the concentration of EVs and the volume of EV solution.

In vitro T cell proliferation Assay. EV gel, EV gel+CpG, EV gel/CpG, and OVA+CpG were placed in a 96-well plate containing 50,000 DCs per well. 100,000 CFSE-stained OT-I cells were added to the surface of the gel, allowing gels to absorb the T cell-containing medium. Finally, T cell medium without IL-2 was added and incubated for 3 days prior to the flow cytometry analysis.

In vivo stability of EV gels. Azido-labeled EVs were incubated with DBCO-Cy5 for 30 min and washed with PBS via ultracentrifugation with an Amicon centrifugal filter (100 kDa). Then, EV gels were formed in vivo by injecting the mixture of Cy5 conjugated azido-labeled EVs with DBCO-PEG into the flank of Balb/c mice. IVIS imaging was used to monitor the release of Cy5-conjugated EVs at different time points.

Immune cell profile of EV gels. C57BL/6 mice were subcutaneously injected with the mixture of azido-labeled EVs and DBCO-PEG. Gels were isolated from mice after 3 days, disrupted, and passed through a 40 μm strainer to collect the cells. Cells were then washed with PBS, stained with fluorophore-conjugated antibodies, and run on a flow cytometer.

Interaction of BMDCs with EV gels. Bone marrow-derived DCs were stained with Calcein AM (10 μM) for 30 min and washed twice with PBS. After washing, cells were added to the top of the EV gel (4 mm diameter×2 mm height) and incubated at 37° C. The viability and proliferation of DCs on top of the gel were monitored over a week using fluorescence microscopy. Pictures of EV gels were taken over time.

Mechanical characterization of EV gels. All tests were performed on an AR-G3 rheometer (TA Instruments) using a 20 mm plate (1.4 mm thickness). The strain sweep test was performed under 0.1-100% strain at a frequency of 1 Hz to determine the linear viscoelastic region of EV gels at different temperatures to ensure that the strain value falls within the linear elastic region. No pre-stress or pressure was applied to the EV gels during the measurement. Solutions of azido-labeled EVs and DBCO-PEG were mixed right before depositing onto the surface plate of the rheometer for the measurement of gelation temperature and time. The gelation temperature of hydrogels for different concentrations of EVs was measured via oscillatory temperature ramp rheological experiments. The storage and loss moduli of formed EV gels were measured by running the time sweep test at 0.5% strain and 1 Hz frequency under room temperature. The viscosity of hydrogels was determined using the flow sweep test performed at 0.5% strain. The stress relaxation of gels was evaluated by subjecting gels to a constant 15% strain.

Release of OVA from EV gel. EV gels (7×10⁸ EVs) were incubated at 37° C. for up to 2 weeks in a 48-well plate. 200 μL aliquots of media were collected on days 1, 3, 5, 9, and 14, and sampled for ELISA assay to quantify the released OVA from EV gels.

Proteomic Analysis. EVs were collected and purified via ultracentrifugation with an Amicon centrifugal filter (100 kDa) and qEV column. Reduction, alkylation of cysteines, and sequential digestion by LysC and trypsin were performed followed by StageTip desalting. Proteins were analyzed on an LC-MS.

Western blot analysis of EVs. EVs were obtained from E.G7-OVA cell culture medium, with or without Ac₄ManAz, and subsequently purified using ultracentrifugation (4347×g, 40 min) and a qEV column. The purified EVs were lysed and assessed for protein content using a BCA assay kit (Sigma, USA). Laemmli sample buffer was added to the lysates, followed by boiling at 100° C. for 5 min. Subsequently, 10 μg of proteins were loaded and electrophoresed on a 12% acrylamide gel (Tris-Glycine/SDS running buffer). The protein bands were then transferred to a PVDF membrane (transfer buffer: 25 mM Tris base, 190 mM glycine, and 20% methanol), and the membrane was stained with an HRP-conjugated OVA polyclonal antibody. EVs derived from B16-F10 cells served as the negative control. EVs were also stained with anti-Tsg101 and anti-CD63 and HRP-conjugated secondary antibody, and visualized using chemiluminescence.

Endocytosis of EVs by DCs. DCs were seeded on coverslips at a density of 6×10⁵ cells and allowed to attach overnight. Cells were incubated with EV-Cy5 for 30 mins or 4 h and washed with PBS. Cells were stained with Rab5 antibody at a dilution 5 of 1:50 and FITC-conjugated Goat anti-rabbit IGG (H+L) or Alexa Fluor 488 conjugated LAMP-1 monoclonal antibody at 1:100 in 0.1% BSA and incubated at 4° C. followed by washing with PBS and fixing the cells with 4% paraformaldehyde solution. Nuclei were stained with DAPI at 1:5000 dilution followed by multiple washes with PBS. The coverslips were mounted onto microscopic slides and imaged using a confocal laser scanning microscope.

ELISA Assay. 96-well high binding ELISA plates were coated with 200 μL of pure OVA protein (0.1 ng/mL to 100 ng/mL) and EV samples (serial dilution from 1/2, 1/10 to 1/10000) in coating buffer (3 replicates each) for overnight at 4° C. The plate was washed with PBST three times followed by the addition of the blocking buffer (5% non-fat milk in PBST) to each well and incubated for 2 h at room temperature in a shaking platform. The plate was washed with PBST 3 times. 50 μL of horseradish peroxidase (HRP) conjugated ovalbumin polyclonal antibody (1:5000) in blocking buffer was added to each well and incubated at room temperature for 2 h. 100 μL of substrate was added to each well, allowing the color to change for 15 min. Finally, 50 μL of stop solution (2 M sulfuric acid) was added to terminate the enzymatic reaction. The optical density was measured at a wavelength of 405 nm using a plate reader.

Immune cell analysis in fibrotic tissue. EV/CpG gels (7×10⁸ EVs and 12.72 ng of CpG) or EV gels (7×10⁸ EVs) were subcutaneously injected to C57BL/6 mice on day 0. On day 3 or day 7, the fibrotic capsule surrounding the gels was harvested for immune cell analysis. Fibrotic tissues were disrupted, digested, and passed through cell strainers, stained with fluorophore-conjugated antibodies, and analyzed using a flow cytometer.

Anti-OVA IgG titer detection. C57BL/6 mice were subcutaneously injected with EV/CpG gel (7×10⁸ EVs and 12.72 ng of CpG), EV gel (7×10⁸ EVs), the mixture of EV (7×10⁸ EVs), DBCO-PEG, and CpG (12.72 ng), or PBS on day 0. Blood samples were collected on day 5, 7, 12, and 18 respectively. Blood was centrifuged at 2,000 g for 5 min to collect the serum, which was stored at −80° C. until further use. For the detection of anti-OVA IgG titers, 96-well high-binding ELISA plates were coated overnight at 4° C. with OVA (20 μg/mL, 50 μL per well) and were further blocked with the blocking buffer for 2 h at room temperature in on shaking platform. After 2-3 washing steps with PBST, diluted serum samples (1/10, 1/100, 1/1000, 1/10,000, 1/100,000) were added to triplicate wells. The plates were then incubated at room temperature for 2 h followed by washing with PBST 10 times. Biotinylated goat anti-mouse-IgG secondary antibody (1:500) in blocking buffer was added to each well and incubated at room temperature for 2 h. After washing with PBST 8 times, HRP-streptavidin (1:500) in PBST was added to each well and incubated for 1 h at room temperature. Peroxidase activity was determined by adding 100 μL of the substrate solution to each well for 30 min. Finally, 50 μL of stop solution (2 M sulfuric acid) was added to terminate the enzymatic reaction. The color was quantified by using a plate reader at 405 nm. The anti-OVA IgG titer was determined by serum dilution.

Blood Test: C57BL/6 mice were subcutaneously injected with an EV/CpG gel (containing 7×10⁸ EVs and 12.72 ng of CpG) or an EV gel (containing 7×10⁸ EVs) on day 0. Blood samples were collected via the retro-orbital method at days 3, 6, and 9 post-injection of EV gels. White blood cell (WBC), red blood cell (RBC), lymphocyte, monocyte, and granulocyte counts were assessed by staining the cells with anti-TER119, anti-CD45, anti-CD3, anti-CD15, anti-CD19, anti-CD14, anti-CD41, and anti-Ly6C, followed by flow cytometry analysis.

Cytokine Analysis. The levels of inflammatory cytokines at the injection sites of EV gel or EV/CpG gel were assessed using a proteome profiling assay (Proteome Profiler Mouse Cytokine Array Kit, Panel A). In brief, C57BL/6 mice received subcutaneous injections of either an EV/CpG gel (containing 7×10⁸ EVs and 12.72 ng CpG) or an EV gel (containing 7×10⁸ EVs). Three days after the injection, the supernatant of cells collected near the gel injection site was processed for cytokine analysis. To prepare the samples, the collected medium was centrifuged to eliminate dead cells and debris. Reagents were prepared in accordance with the manufacturer's instructions (R&D, USA). The membranes were initially blocked by incubating with Array Buffer 6 for 1 hour on a shaker. Each sample was diluted by adding 1 mL of the collected medium to 0.5 mL of Array Buffer 4, and the volume was adjusted to 1.5 mL with Array Buffer 6. Then, 15 μL of the mouse cytokine detection antibody cocktail was added to each sample and incubated for 1 hour. Following the incubation, the membranes were washed and incubated with the antibody-sample mixture overnight at 4° C. On the following day, the membranes were washed again and incubated with Streptavidin-HRP (1:2000) for 30 min at room temperature. After a final wash, 1 mL of Chemi Reagent mix was applied to each membrane, which was then exposed to a chemiluminescence imager for 2-5 min. Positive signals on the membranes were identified using a transparency overlay template provided by the manufacturer, aligning it with the reference spots for each cytokine. Pixel densities (average signals from duplicate spots) were analyzed using ImageJ software, with background signal subtracted from the average signal.

Vaccination and prophylactic study. C57BL/6 mice were divided into five groups: EV/CpG gel (7×10⁸ EVs and 12.72 ng of CpG), EV gel+CpG (7×10⁸ EVs and 12.72 ng of CpG), OVA (50 μg) and CpG (50 ng), EV gel (7×10⁸ EVs), or PBS. EV gels or the solution of OVA and CpG or PBS were subcutaneously injected into the flank of mice on day 0. Blood was drawn on days 6, 9, and 14 for the analysis of SIINFEKL (SEQ ID NO: 1)-specific CD8⁺ T cells in PBMCs. For tetramer analysis, PBMCs were stained with fluorophore-conjugated SIINFEKL (SEQ ID NO: 1) tetramer, anti-CD3, anti-CD8, and e780 fixable viability dye for 20 min, and analyzed on a flow cytometer. For IFN-γ restimulation, PBMCs were treated with SIINFEKL (SEQ ID NO: 1) peptide for 1.5 h and Golgi plug for 2.5 h, and then stained with fluorophore-conjugated anti-CD3, anti-CD8, and e780 fixable viability dye. Cells were further treated with the fixation & permeabilization buffer and stained with fluorophore-conjugated anti-IFN-γ for flow cytometry analysis. On day 15, E.G7-OVA tumor cells (100k cells in 50 μL of HBSS) were subcutaneously injected into C57BL/6 mice. The tumor volume and body weight of the mice were measured twice a week. Tumor volume was calculated using the formula L×W²/2 (L=long axis diameter; W=short axis diameter). The endpoint was defined as the day the largest diameter of tumors reaches 20 mm, or animals become moribund, or animals have a body weight loss of >20%, or animals have a body condition of 2 or less on a 5 point scale.

Therapeutic tumor study of EV hydrogel vaccines. E.G7-OVA cells (500k cells in 50 μL of HBSS) were subcutaneously injected into the right flank of C57BL/6 mice on day 0. When the tumors reached a diameter of 5-7 mm, the mice were randomly divided into five groups: EV/CpG gel+α-PD-1, EV/CpG gel, EV gel, α-PD-1, or PBS. Gels were injected on day 9. α-PD-1 (100 μg per dose) was intraperitoneally injected on days 13, 16, and 19. The tumor volume and mouse body weight were measured 2-3 times a week. Tumor volume was calculated using the formula L×W²/2 (L=long axis diameter; W=short axis diameter). The endpoint was defined as the day the largest diameter of tumors reaches 20 mm, or animals become moribund, or animals have a body weight loss of >20%, or animals have a body condition of 2 or less on a 5 point scale.

Tumor Microenvironment Analysis. C57BL/6 mice were assigned to seven experimental groups: EV/CpG gel (7×10⁸ EVs and 12.72 ng CpG), EV gel+CpG (7×10⁸ EVs and 12.72 ng CpG), OVA (50 μg) and CpG (50 ng), EV gel (7×10⁸ EVs), EV only (7×10⁸ EVs), unconjugated EV+DBCO-PEG+CpG (7×10⁸ EVs and 12.72 ng CpG), or PBS. On Day 0, E.G7-OVA cells (200,000) were washed with HBSS buffer and subcutaneously injected into the upper right flank of the mice. On Day 12, the respective EV gel formulations were injected subcutaneously. Tumors and gels were harvested on Day 15 for further analysis. For processing, tumors and gels were mechanically disrupted using a syringe plunger, then passed through a 40 μm cell strainer and rinsed with 10 mL of RPMI. The resulting cell suspension was collected, washed with PBS, and stained for flow cytometry analysis.

Statistical analysis. For comparisons between two groups, two-tailed Welch's t-test or two-tailed student's t-test were used. For multiple comparisons, one-way analysis of variance (ANOVA) with a post hoc Fisher's LSD test was used. The results were deemed significant at 0.01<*P≤0.05, highly significant at 0.001<**P≤0.01, and extremely significant at ***P≤0.001.

### Example 2

### Metabolic Tagging of EVs

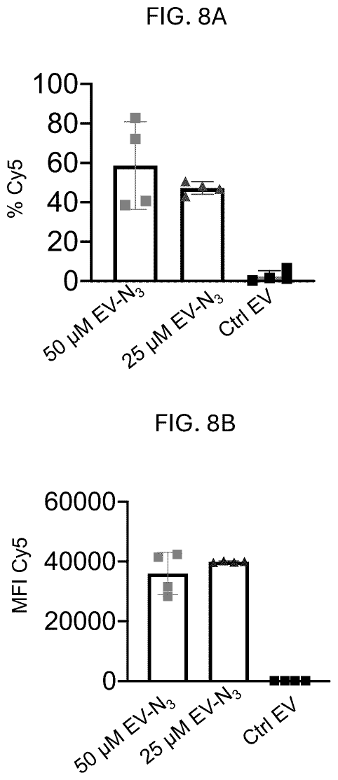

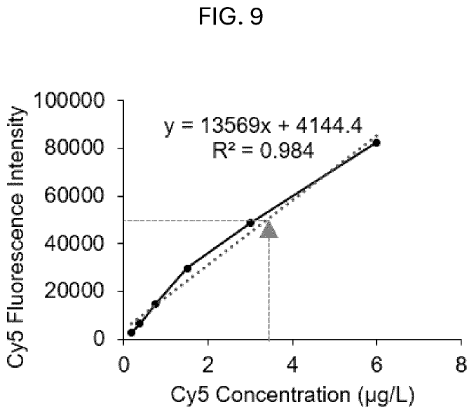

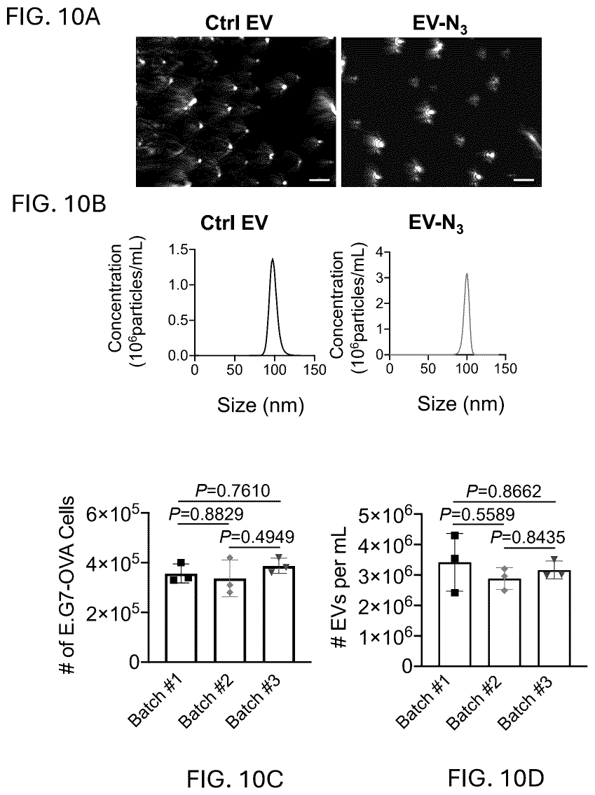

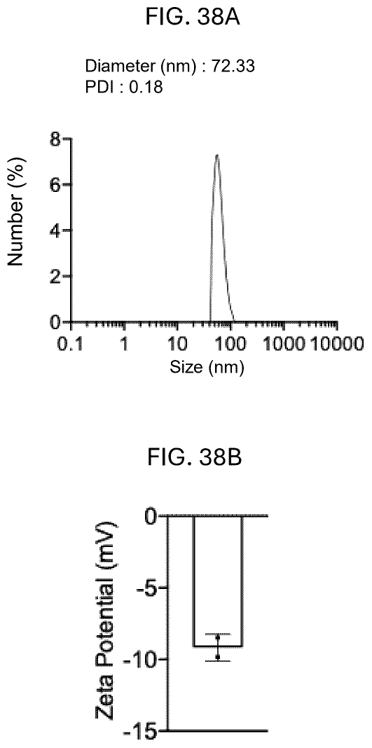

To generate azido-labeled EVs, E.G7-OVA cells were incubated with Ac₄ManNAz, a commonly used metabolic labeling agent, for three days, followed by the collection of EVs via ultracentrifugation (100 kDa MW cutoff) (FIG. 2A). Consistent with previous reports, Ac₄ManNAz was able to metabolically label E.G7-OVA cells with azido groups (FIGS. 8A-8B). To confirm whether azido-labeled E.G7-OVA cells can secrete azido-tagged EVs, EVs from Ac₄ManNAz- or PBS-treated E.G7-OVA cells were incubated with DBCO-Cy5 for 30 min, followed by ultracentrifugation to remove unreacted DBCO-Cy5. Compared to control EVs, EVs from Ac₄ManNAz-treated E.G7-OVA cells showed significantly higher Cy5 fluorescence intensity (FIG. 2B), demonstrating the successful metabolic tagging of EVs with azido groups. By quantifying the fluorescence intensity of conjugated DBCO-Cy5, the surface density of azido groups was estimated to be >4,000 per EV (FIG. 9). The average size and relative concentration of labeled and unlabeled EVs showed negligible differences, as determined by the Nanoparticle Tracking System (NTA) (FIGS. 2C-2D; FIGS. 10A-10B). TEM images also confirmed a similar morphology and size distribution between azido-labeled and unlabeled EVs (FIG. 2E). Dynamic light scattering (DLS) confirmed a similar size distribution and a zeta potential of −9.2 mV (FIGS. 38A-38B). About 10⁹ purified EVs could be obtained after culturing 1 million E.G7-OVA cells for three days. It is noteworthy that a fraction of EVs may come from FBS in the culture medium, and future studies that aim to obtain purer EVs may involve the removal of EVs from fetal bovine serum (FBS) prior to cell cultures.

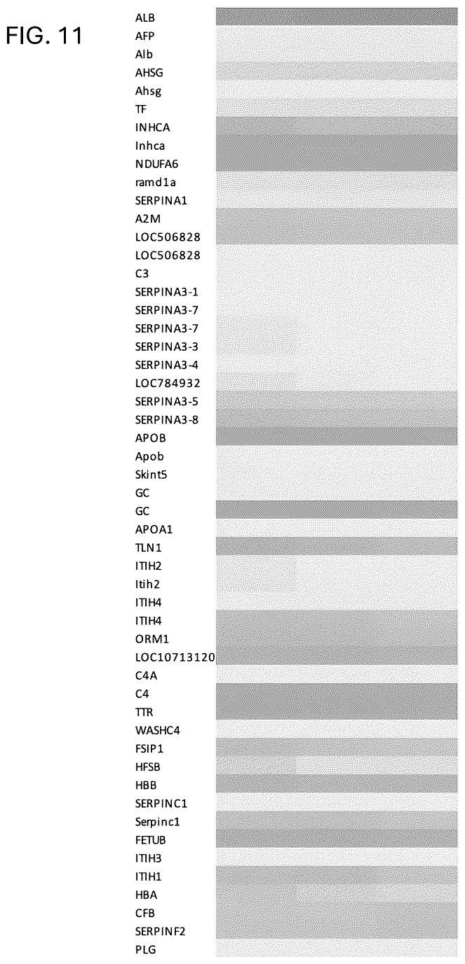

It is noteworthy that ˜10⁹ purified EVs could be obtained after culturing 1 million E.G7-OVA cells for three days. We also verified the batch-to-batch consistency in harvesting EVs from E.G7-OVA cells (FIGS. 10C-10D). Proteomic analysis confirmed the presence of various types of proteins in EV and azido-labeled EV (FIG. 11). Also, the protein composition between different batches of EVs was consistent (FIG. 11). These experiments demonstrated that metabolic glycan labeling of the parent cells can generate chemically tagged EVs without altering the biogenesis, morphology, and size of EVs.

### Example 3

### EV Hydrogels with Tunable Mechanics and Gelation Temperature

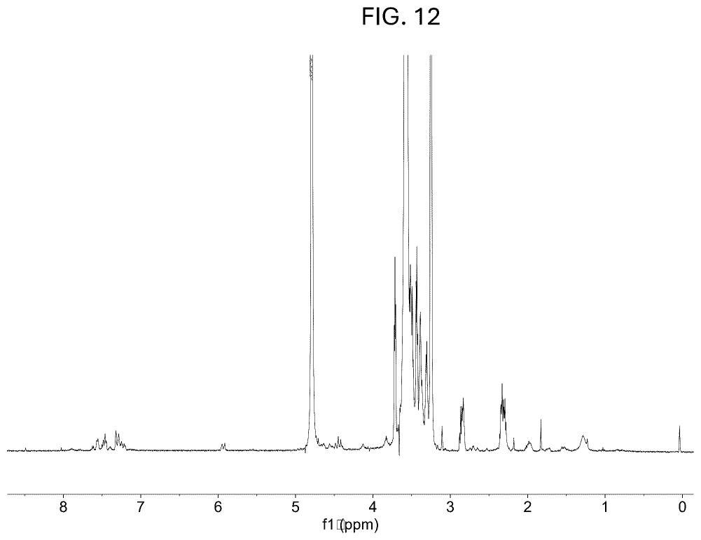

To fabricate EV hydrogels, we functionalized 8-arm PEG with DBCO (FIG. 12) and mixed the synthesized DBCO-PEG with azido-labeled EVs at 37° C. (FIG. 2F). The mixture rapidly formed a hydrogel as a result of the click reaction between DBCO and azido groups (FIG. 2G). In contrast, the mixture of unlabeled EVs and DBCO-PEG failed to form a hydrogel even after days (FIG. 2G). The formed hydrogel has a storage modulus (G′) of ˜13.5 kPa and loss modulus (G″) of ˜2 kPa (FIGS. 2H-2I). We also performed SEM imaging of the formed EV hydrogels, which clearly revealed a crosslinked network of nanosized EVs (FIG. 2J). The EVs were tightly packed and formed a nanoporous gel network (FIG. 2J). These experiments demonstrated that EV hydrogels can be formulated via click chemistry-mediated crosslinking between azido-labeled EVs and DBCO-PEG.

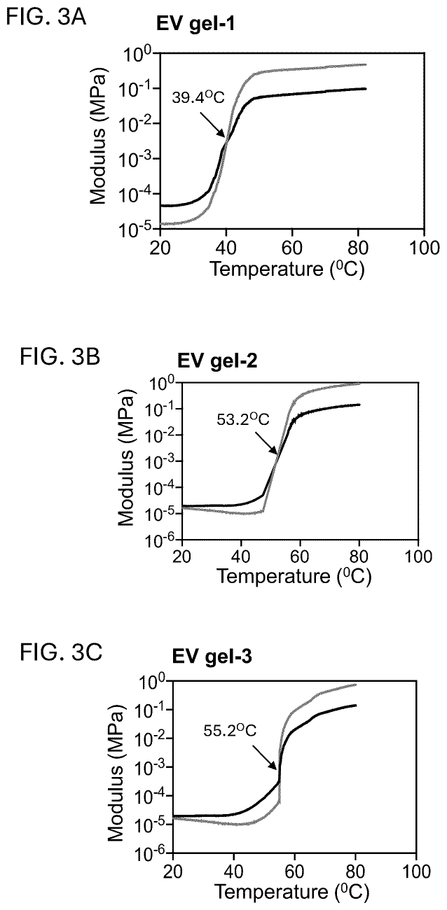

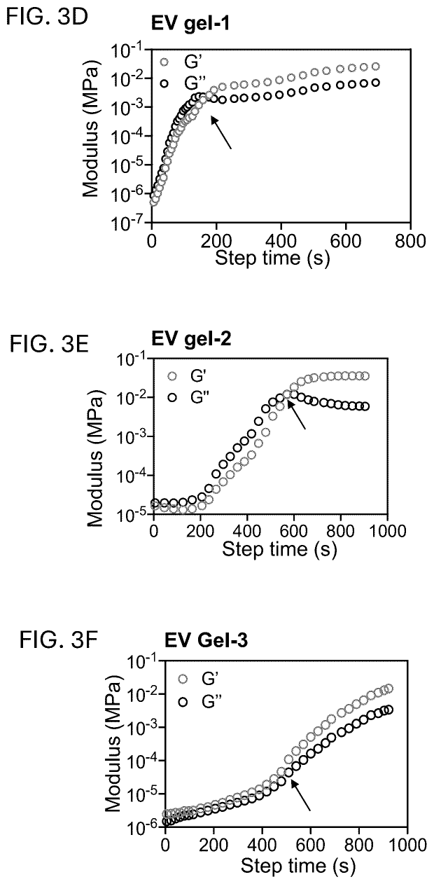

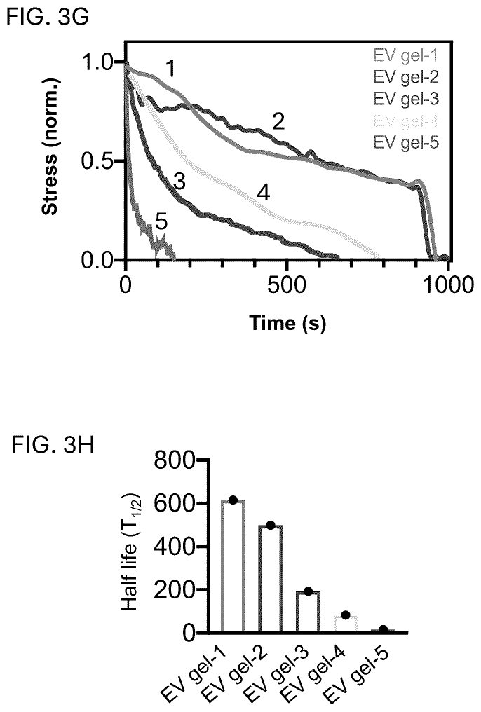

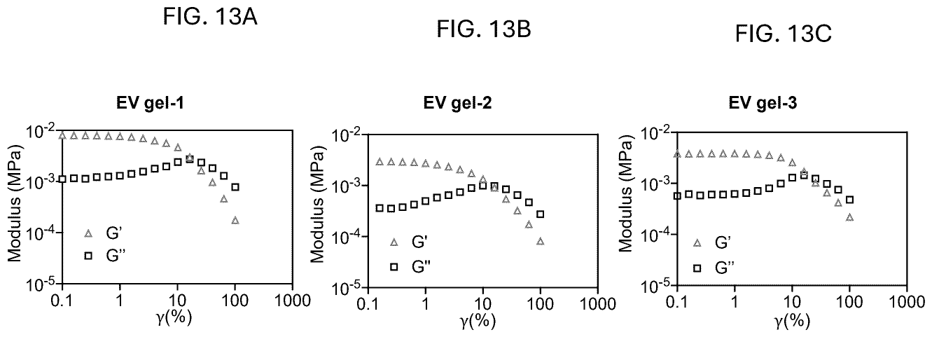

To better understand the EV hydrogel system, we next studied the gelation process and mechanical properties of EV hydrogels formed with varying concentrations of azido-labeled EVs. The final concentration of DBCO-PEG was kept at 20 mg/mL, while the final concentration of EVs was set at 7×10⁹/mL, 1.75×10⁹/mL, 1.17×10⁹/mL, 0.875×10⁹/mL, and 0.70×10⁹/mL for EV gel-1, EV gel-2, EV gel-3, EV gel-4, and EV gel-5, respectively. We first analyzed the gelation temperature of EV hydrogels via dynamic temperature ramp measurements, which showed a decreased gelation temperature for higher EV concentrations (FIGS. 3A-3C). In particular, EV gel-1 formed at an EV concentration of 7×10⁹/mL and DBCO-PEG concentration of 20 mg/mL has a gelation temperature of 39.4° C. (FIG. 3A). This gelation temperature (slightly higher than body temperature) is especially useful for in vivo applications when the mixture of azido-labeled EVs and DBCO-PEG can stay as a solution below the body temperature but forms a gel once injected into the body. By fixing the temperature at 37° C., higher EV concentrations resulted in more rapid gelation processes and higher storage moduli as expected (FIGS. 3D-3F; FIGS. 13A-13C). EV gels also exhibited a viscoelastic behavior, with the relaxation half-life increasing with the concentration of EVs (FIGS. 3G-3H). These experiments demonstrated the tunability of the gelation temperature, gelation time, storage moduli, and relaxation half-lives of EV hydrogels by varying the concentration of azido-labeled EVs.

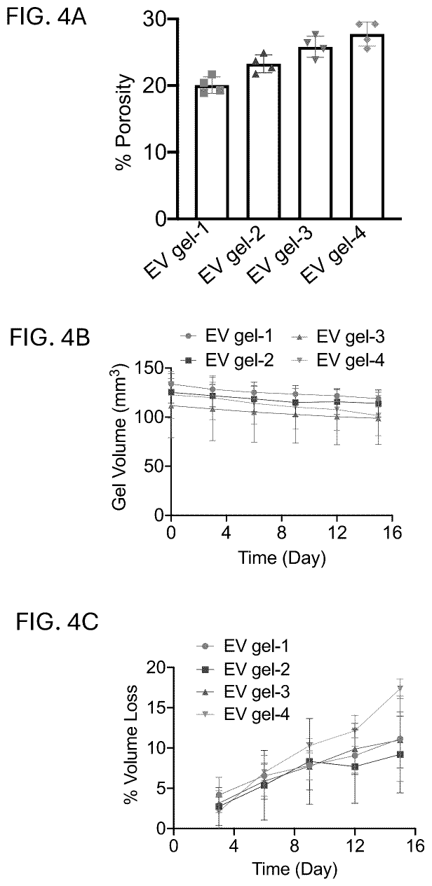

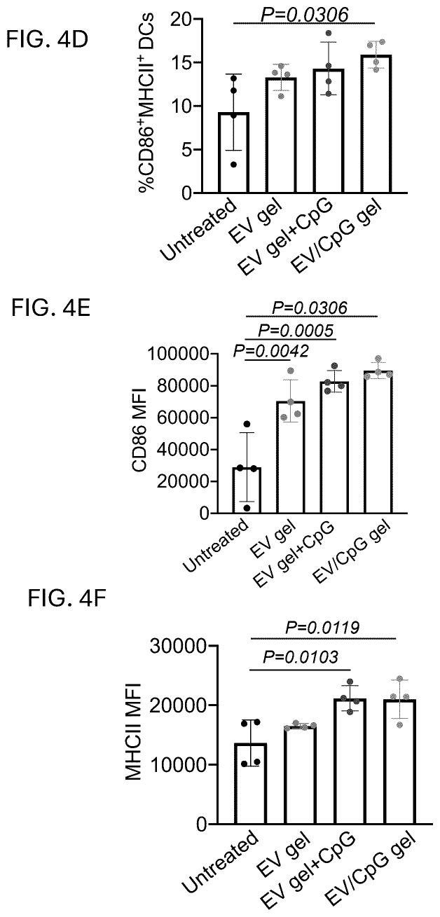

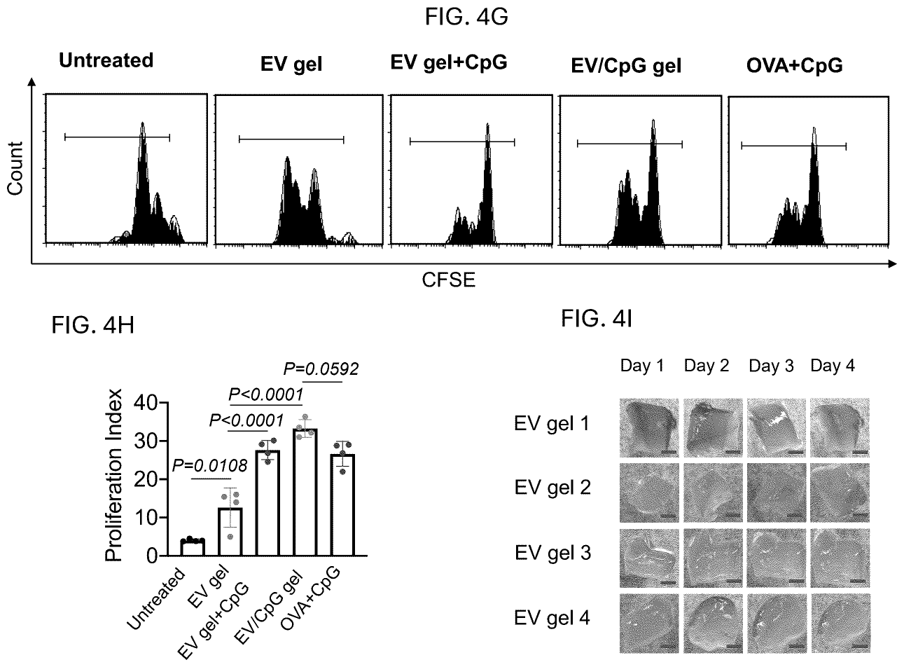

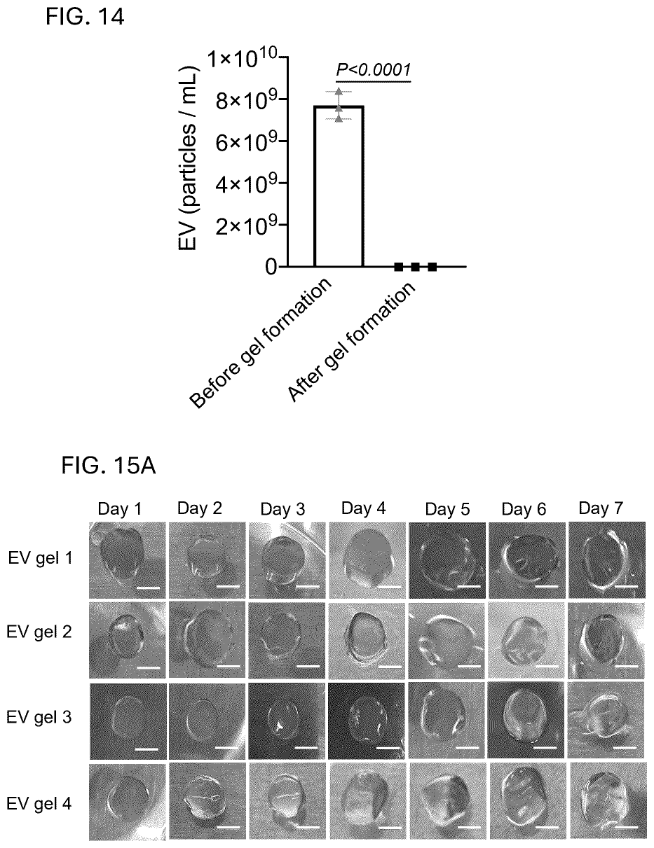

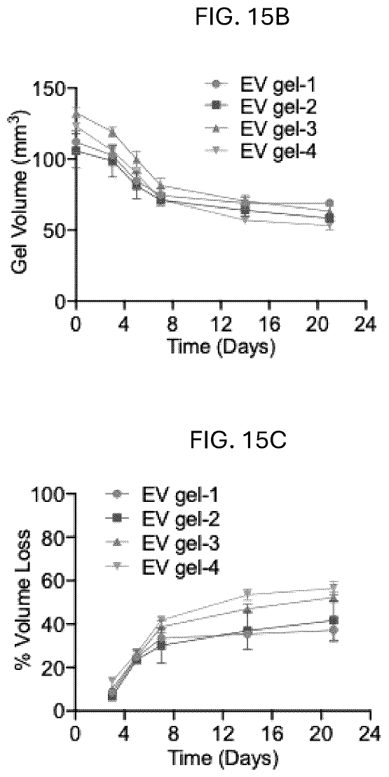

It is noteworthy that EV gel-1 showed a high EV capturing efficiency (>99%) during the gel formation process (FIG. 14). Consistent with the mechanical properties, EV gels with a decreased EV concentration exhibited a higher porosity (FIG. 4A). By immersing EV gels in PBS, EV gel-1 showed a ˜10% volume loss over 15 days and EV gel-4, which is assumed least stable, showed a ˜17% volume loss over 15 days (FIGS. 4B-4C; FIG. 15), demonstrating the overall good stability of EV hydrogels. We envision the slow degradation of EV hydrogels would enable the long retention of EV gels while allowing for the gradual release of EV-encased contents over time. Among all gels, EV gel-1 with rapid gelation at 37° C., favorable mechanical properties, and good stability was used for subsequent experiments.

### Example 4

### Processing of EV Hydrogel-Encased Antigens by DCs

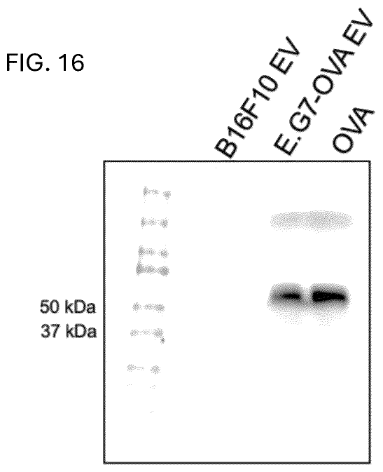

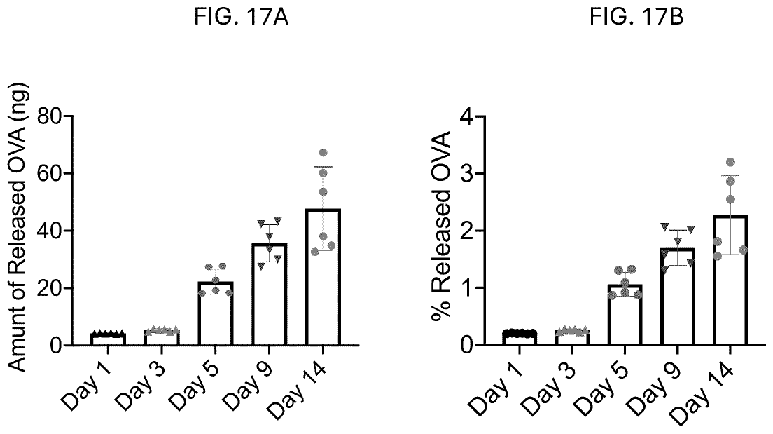

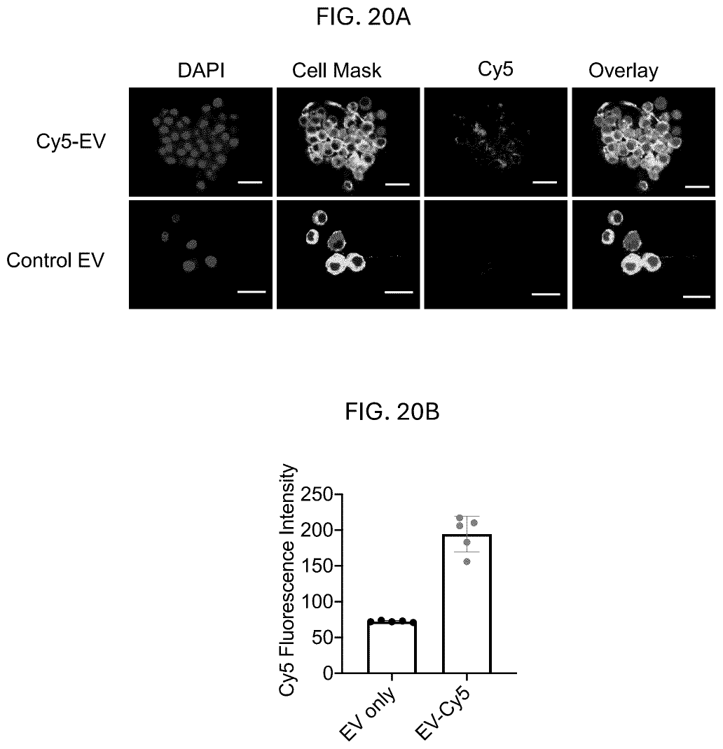

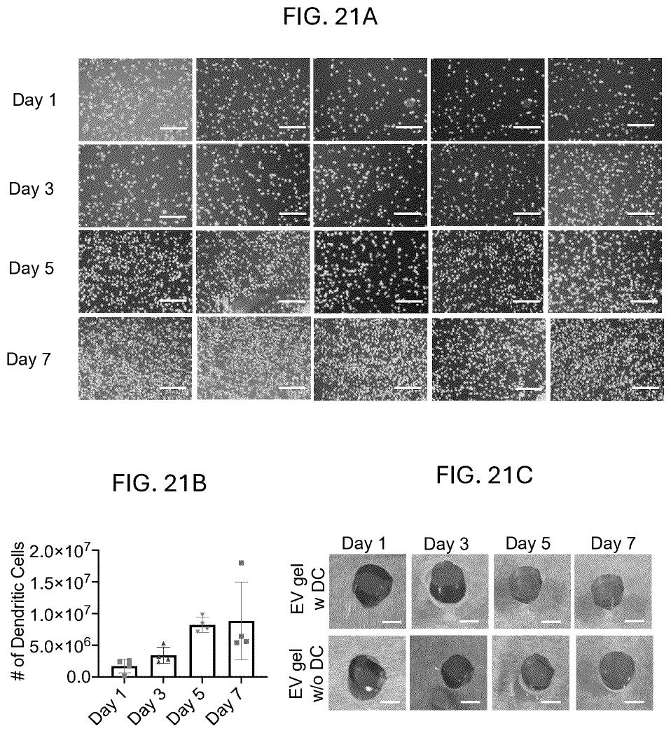

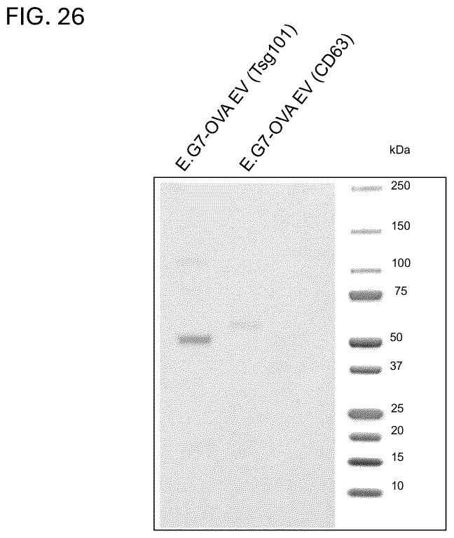

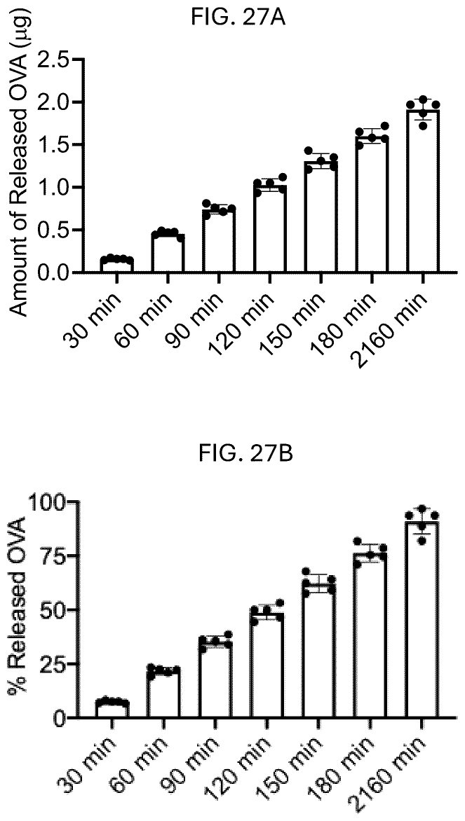

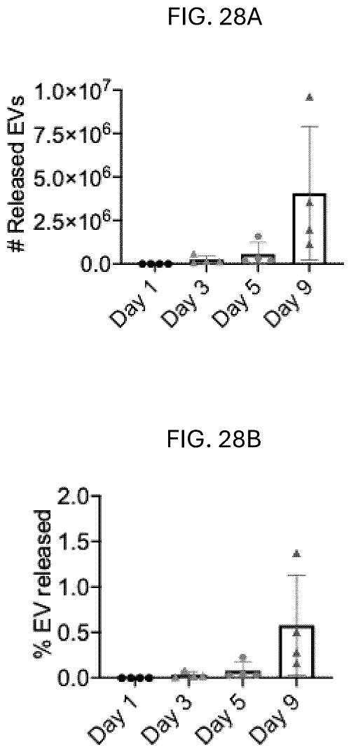

Next, we studied the interaction between DCs and EV hydrogels, and the processing and presentation of EV gel-encased antigens by DCs. We first confirmed the presence of OVA protein in E.G7-OVA derived EVs via western blot (FIG. 16). We have also confirmed the presence of Tsg101 and CD63, two common EV markers, in E.G7-OVA derived EVs (FIG. 26). Per the ELISA assay, the density of OVA protein was 2.9×10⁻⁹ μg per EV. Azido-labeled EVs were mixed with DBCO-PEG at 37° C. for 3 min to form the EV gel. We analyzed the release kinetics of OVA protein from the EV gel by quantifying the released OVA in the culture media at different times using ELISA assay. About 48 ng of OVA was released from EV gels over the course of 14 days (FIG. 17A), which accounts for ˜2.3% of the loaded OVA (FIG. 17B), demonstrating the stability of EV gels and sustained release of EV-encased molecules over time. OVA shows a burst and much faster release from conventional alginate hydrogel (FIGS. 27A-27B). We also analyzed whether EVs could be released from the EV gels by incubating EV gels in PBS at 37° C. and quantifying the number of particles in PBS over time via NTA. Few particles were detected within 5 days and ˜0.5% of initially loaded EVs were detected by day 9 (FIGS. 28A-28B), indicating that the vast majority of EVs remain in the gels and gradually release the encased molecules over time. To assess the interaction between EV gels and DCs, we stained bone marrow-derived DCs (BMDCs) with Calcein AM, added them to the top of the EV gel, and monitored BMDCs and EV gels over time. EV gels remained stable for over 7 days (FIGS. 21A-21C). BMDCs mostly remained on the surface of the EV gels and exhibited good viability and proliferation over time (FIG. 21A-21B). We also confirmed the rapid uptake of EVs, via conjugation of DBCO-Cy5 to azido-labeled EVs, by DCs (FIG. 20A). Per confocal imaging, Cy5-conjugated EVs partially overlaid with the early endocytic marker Rab5 at 30 min and late endocytic marker LAMP-1 at 4 h (data not shown). Flow cytometry analysis also confirmed the uptake of Cy5-conjugated EVs by DCs (FIG. 20B).

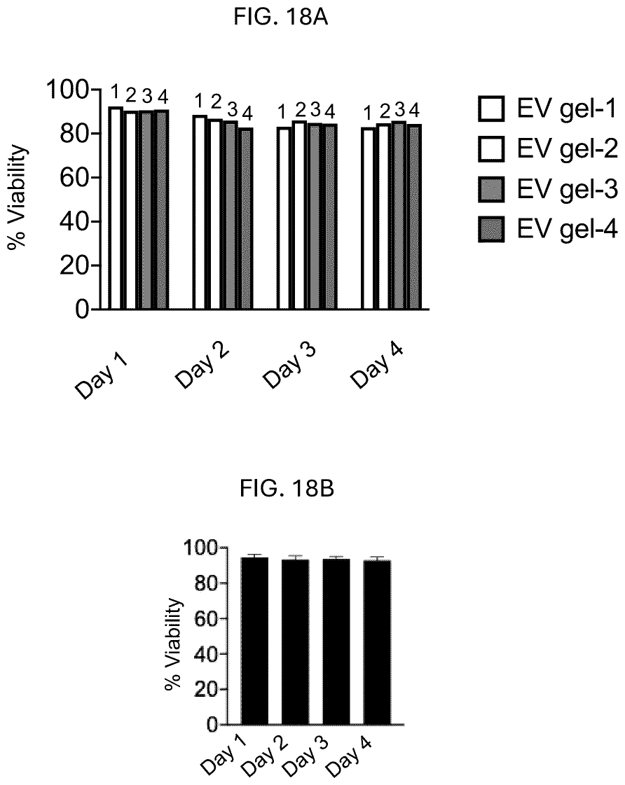

To better activate DCs, CpG adjuvant was incorporated into EV gels via either physical encapsulation of free CpG (EV gel+CpG) or covalent conjugation of DBCO-CpG (EV/CpG gel). Bone marrow-derived DCs (BMDCs) were loaded into the EV gel, CpG-encapsulating EV gel (EV gel+CpG), or CpG-conjugated EV gel (EV/CpG gel) and incubated for 16 h. Compared to untreated DCs, DCs in EV gels expressed a higher level of CD86 (FIGS. 4D-4E), and DCs in CpG-loaded EV gels showed a further increased CD86 expression (FIGS. 4D-4F). It is noteworthy that DCs loaded into EV gels showed high viability over time (FIG. 18A).

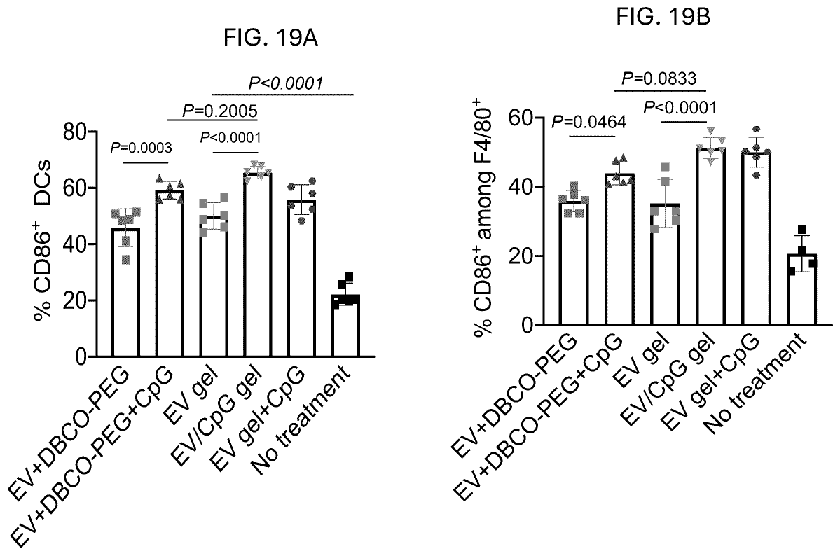

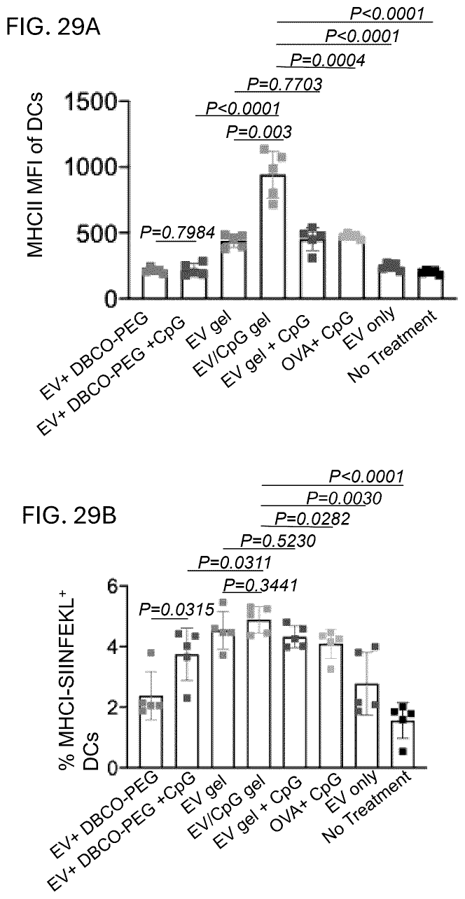

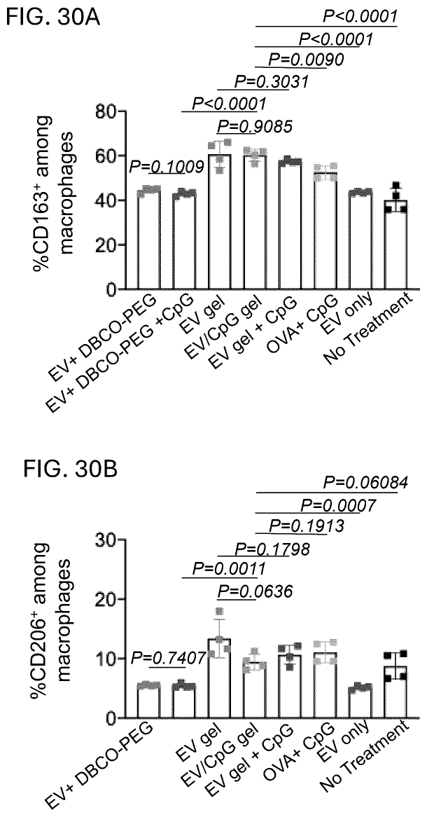

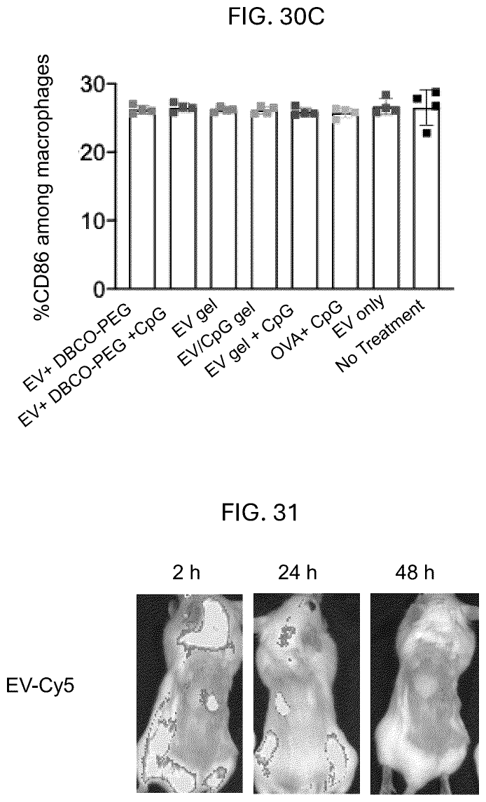

To study DC activation, we seeded BMDCs on top of the EV gel, CpG-encapsulating EV gel (EV gel+CpG), or CpG-conjugated EV gel (EV gel conjugated with DBCO-CpG, denoted as EV/CpG gel), or incubated DCs with a solution of non-crosslinked EVs (with or without CpG). Compared to untreated DCs, DCs treated with EV gels showed an upregulated expression of CD86 (FIG. 19A). The incorporation of CpG, either via physical encapsulation or covalent conjugation, further increased CD86 expression on DCs (FIG. 19A). Compared to the solution of non-crosslinked EVs and CpG, CpG-loaded EV gels showed a similar DC activation effect (FIG. 19A). Similarly, CpG-conjugated EV gels also managed to upregulate the expression of MHCII and MHCI-SIINFEKL (SEQ ID NO: 1) complexes on DCs, in comparison with EV gel alone or to the solution of non-crosslinked EVs and CpG (FIGS. 29A-29B). The effect of EV gels on macrophages was also studied by incubating bone marrow-derived macrophages with EV gel, CpG-encapsulating EV gel, CpG-conjugated EV gel, EV alone, or the solution of non-crosslinked EVs and CpG for 16 h, and examining the surface expression of CD163, CD206, and CD86. Compared to the solution of non-crosslinked EVs and CpG, EV gels slightly upregulated the expression of CD163 and CD206 on macrophages (FIGS. 30A-30C). To further understand the interaction of DCs with EV gels, BMDCs were also loaded into the EV gel, CpG-encapsulating EV gel, or CpG-conjugated EV gel, and incubated for 16 h. Compared to untreated DCs, DCs in EV gels expressed a higher level of CD86, and DCs in CpG-loaded EV gels showed a further increased CD86 expression (FIGS. 4D-4F). BMDCs loaded into EV gels showed high viability over time (FIG. 18B). We next studied whether DCs can process and present E.G7-OVA EV-encased antigens within the hydrogel. BMDCs were loaded into the EV gel, CpG-encapsulating EV gel, or CpG-conjugated EV gel, or directly incubated with the solution of OVA and CpG or the solution of EV alone for 16 h, and then co-incubated with CFSE-stained OT-I cells for three days. DCs pretreated with EV gels alone were able to improve the proliferation of OT-I cells compared to non-pretreated DCs (FIGS. 4G-4H), indicating the uptake and processing of EV-encased OVA antigens by DCs. Consistent with the DC activation result, DCs pretreated with CpG-loaded EV gels induced higher proliferation of OT-I cells than DCs pretreated with EV gel alone (FIGS. 4G-4H).

### Example 5

### In Vitro and In Vivo Stability and Immune Cell Profile of EV Hydrogels

We next studied the stability of EV gels. By immersing EV gels in PBS, EV gel-1 showed a ˜10% volume loss after 15 days and EV gel-4, which is assumed least stable, showed a ˜17% volume loss after 15 days (FIGS. 4B, 4C, and 4I), demonstrating the overall excellent stability of EV hydrogels in vitro. We also examined the degradation of EV gels in 10% FBS at 37° C., which showed a 37% volume loss over a course of 21 days for EV gel-1 (FIGS. 15A-15C).

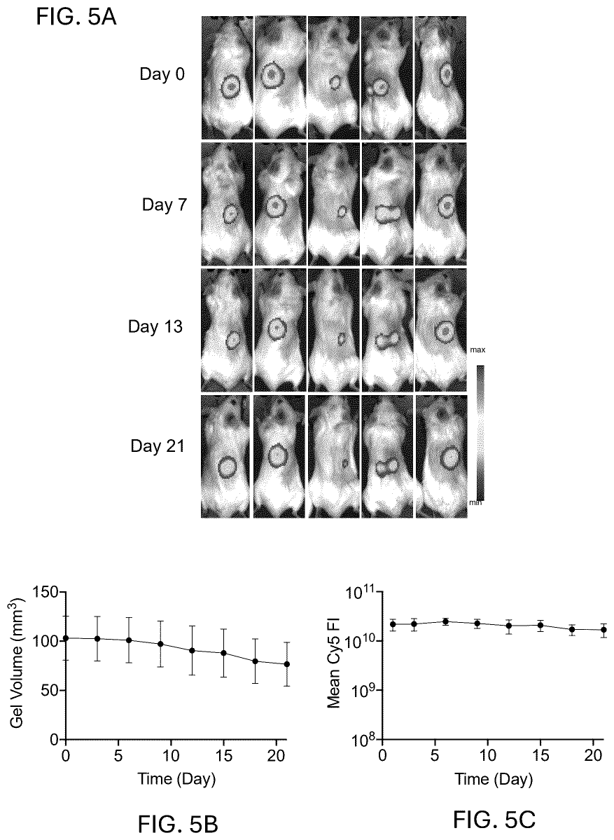

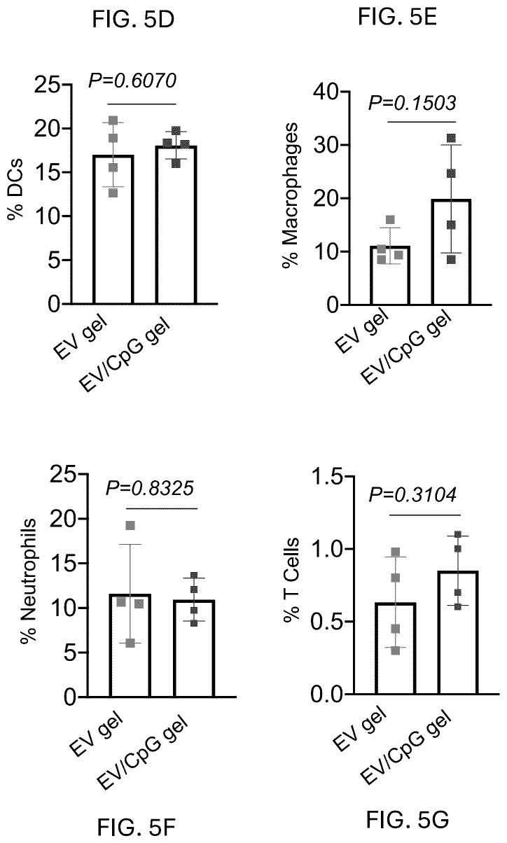

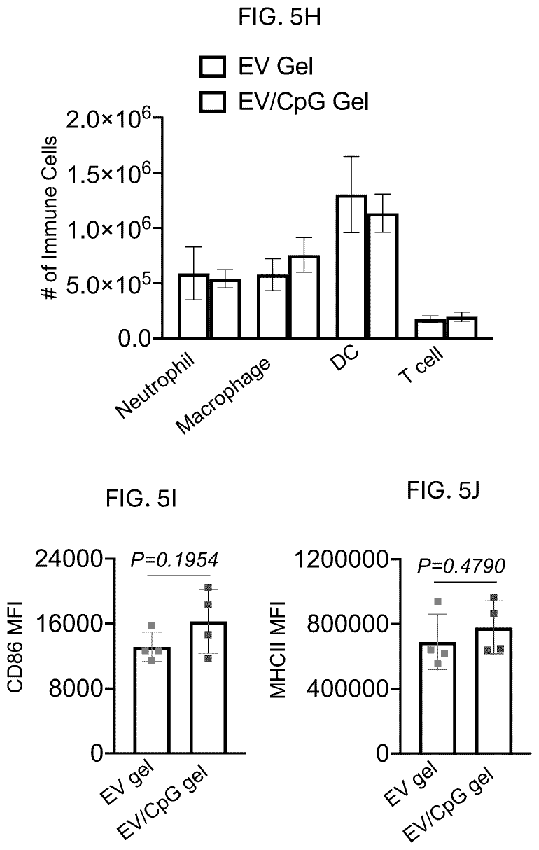

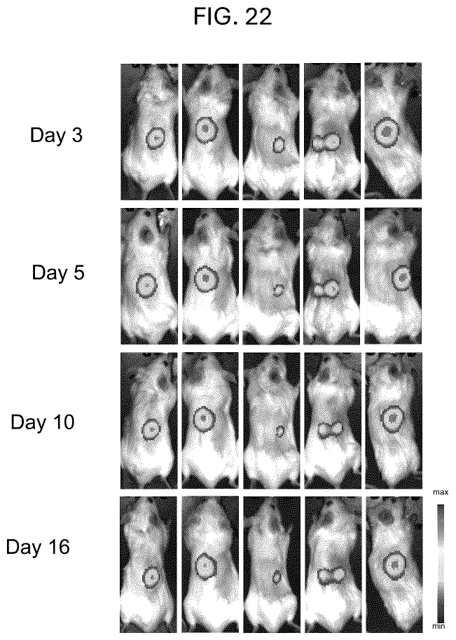

To study in vivo stability of EV hydrogels azido-labeled EVs were conjugated with DBCO-Cy5 and then mixed with DBCO-PEG at room temperature, prior to subcutaneous injection into the flank of Balb/c mice via an 18 G needle. Mice were monitored via IVIS imaging for three weeks (FIG. 5A). The mixture of EVs and DBCO-PEG was in a liquid state at room temperature, but rapidly formed a gel-like bulge after injection into the mice. The in situ formed EV gel showed minimal degradation over the first five days, as evidenced by the negligible changes in the gel volume and Cy5 fluorescence intensity (FIGS. 5A-5c: FIG. 22). Over the course of 21 days, EV gels remained intact at the injection site, despite the gradual decrease of gel volume over time (FIG. 5B). It is noteworthy that Cy5-conjugated EVs alone, upon subcutaneous injection, were rapidly cleared from the injection site within 2 h (FIG. 31). It is possible that the slow degradation of EV hydrogels could enable the long retention of EV gels while allowing for the gradual release of EV-encased contents over time.

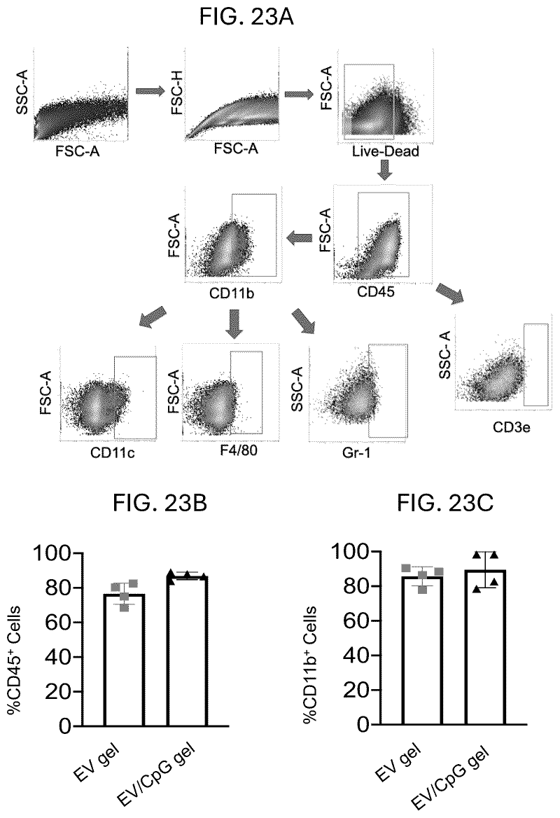

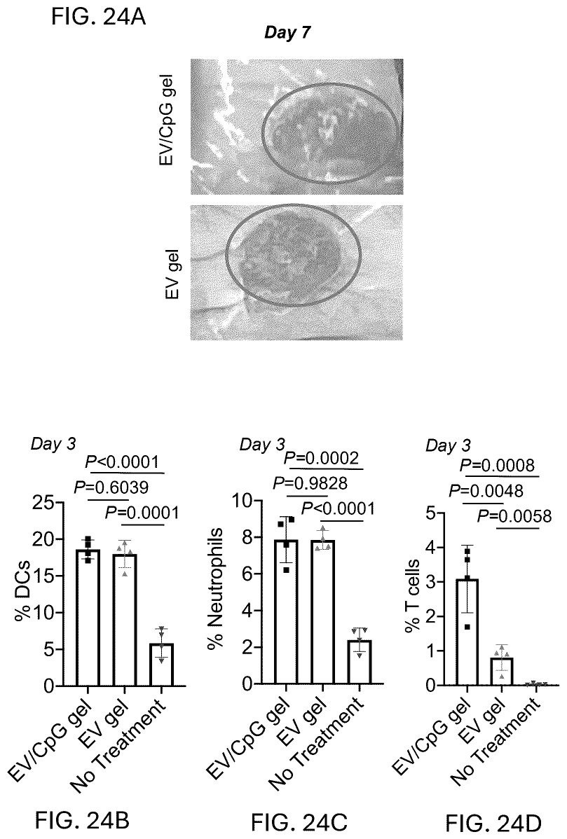

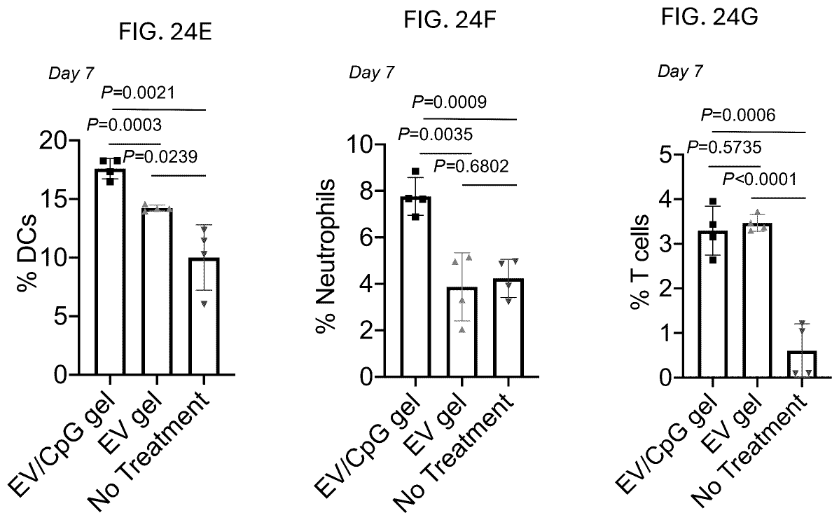

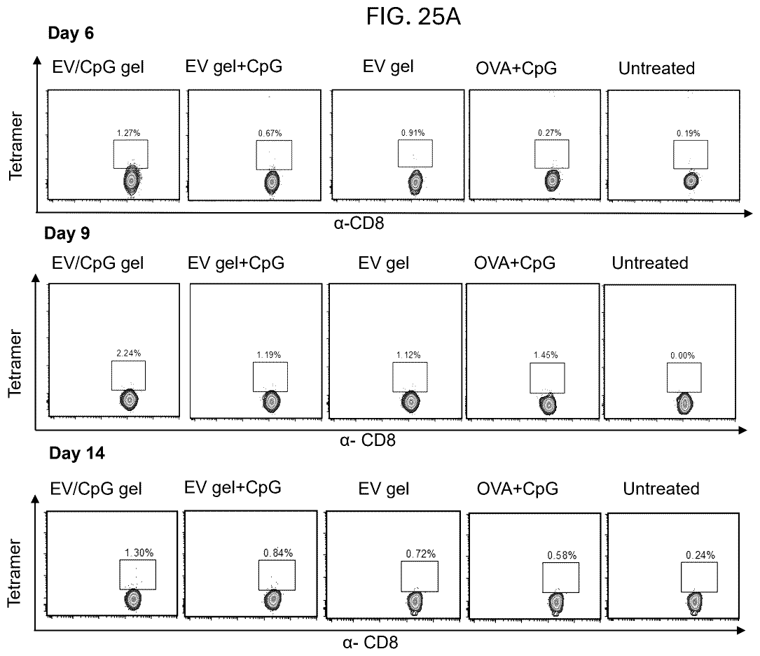

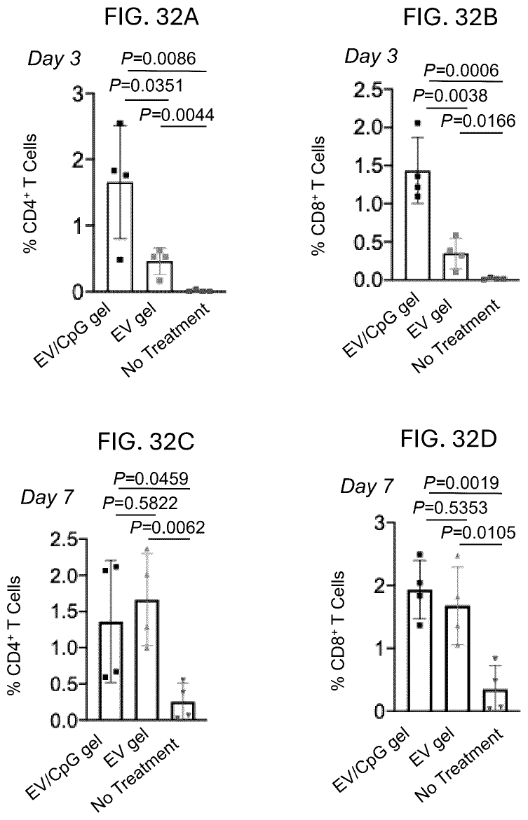

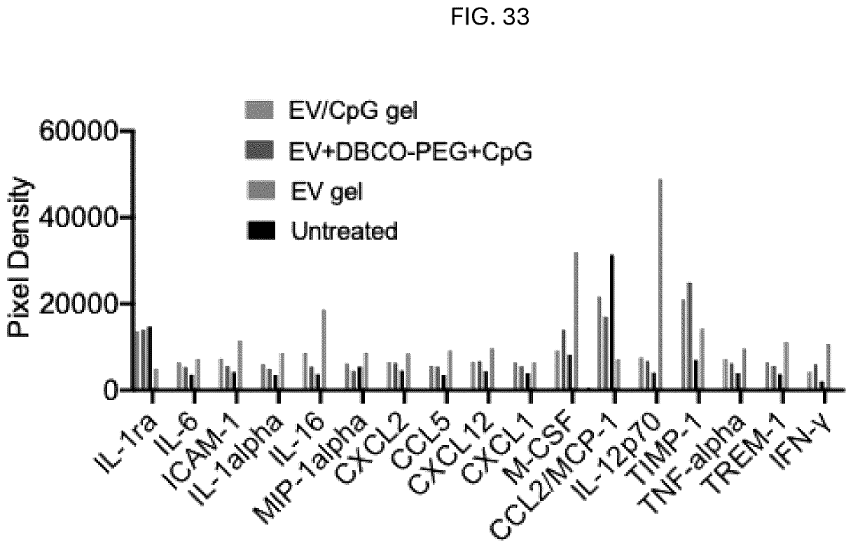

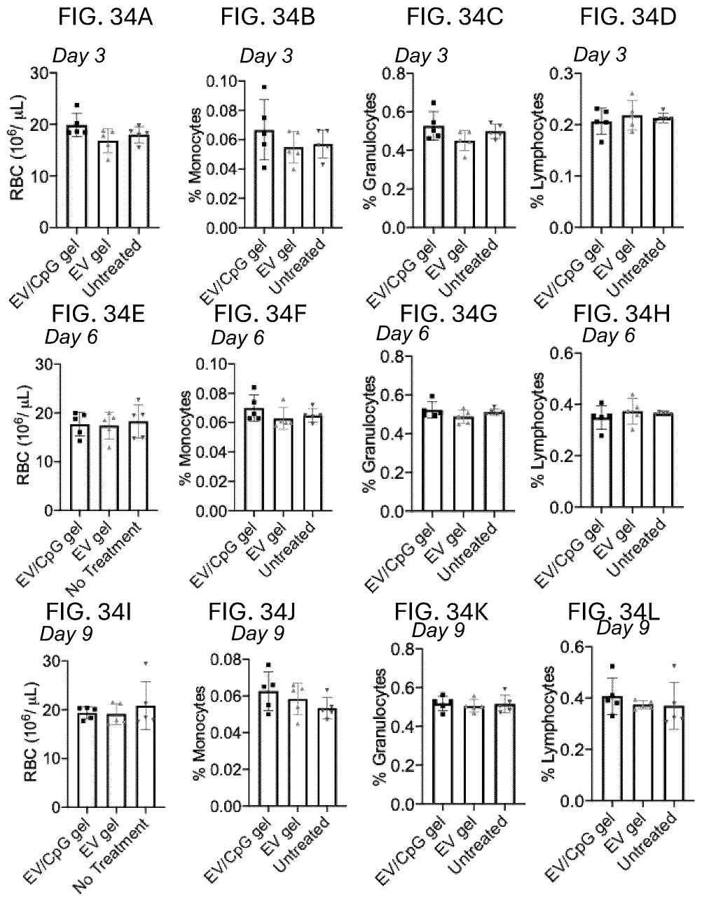

We also analyzed the immune cells in the subcutaneous EV hydrogels at 3 days post the gel injection (FIGS. 23A-23C). Among the CD45⁺ population, ˜17% were CD11c⁺ DCs (FIG. 5D), ˜10% were CD11b⁺F4/80⁺ macrophages (FIG. 5E), ˜10% were CD11b⁺Gr1⁺ neutrophils (FIG. 5f), and ˜0.5% were CD3⁺ T cells (FIG. 5G). Compared to blank EV gels, CpG-conjugated EV gels showed negligible changes in the percentage and number of DCs, macrophages, neutrophils, and T cells at the gel site (FIGS. 5D-5H). DCs in the EV gels or CpG-conjugated EV gels also expressed a similar level of CD86 and MHCII (FIGS. 51-5J). To clarify, most of the immune cells were in the surface layers of EV gels, although a small number of cells could infiltrate into the gels over time. To better understand the inflammatory responses at the gel site, we subcutaneously injected EV/CpG gels or EV gels on day 0. On day 3 or 7, a capsule structure surrounding the EV gel was formed (FIG. 24A). On day 3, the capsule tissue surrounding EV/CpG gels showed a similar population of DCs and neutrophils and a slightly higher population of T cells in comparison with the capsule structure surrounding EV gels (FIGS. 24B-24D). On day 7, the capsule structure surrounding EV/CpG gels showed a slightly higher population of DCs and neutrophils than the capsule structure surrounding EV gels (FIGS. 24E-24G). The number of CD4+ and CD8⁺ T cells showed negligible differences between EV/CpG gel and EV gel (FIGS. 32C-32D). We also measured the cytokine levels at the EV gel injection site on day 3, which showed an increase level of inflammatory cytokines such as TNF-α, IL-16, IL-12, and M-CSF in mice injected with EV/CpG gel or EV gel than untreated mice (FIG. 33). The upregulated level of inflammatory cytokines aligns with the infiltration of DCs, neutrophils, and other immune cells (FIGS. 24E-24G, FIGS. 32A-32D), and likely contribute to the sampling and presentation of EV-encased tumor antigens by antigen presenting cells at the gel site. In a separate study, we also analyzed the blood cells at 3, 6, and 9 days post administration of EV/CpG gel and EV gel, which showed negligible differences in the number of red blood cells, monocytes, lymphocytes, and granulocytes in comparison with the untreated group (FIGS. 34A-34C), demonstrating the minimal effect of EV gel treatment on blood cell populations.

### Example 6

### Enhanced CTL Response of EV Gel Vaccines

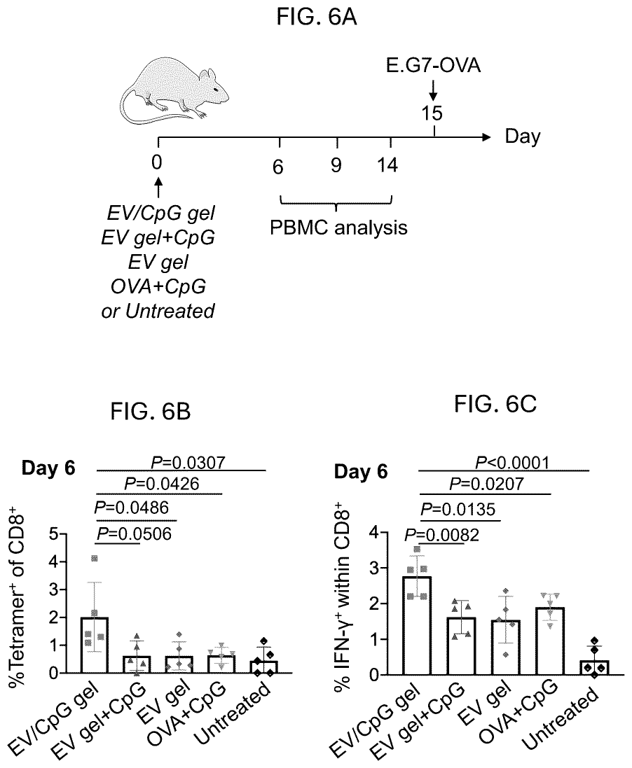

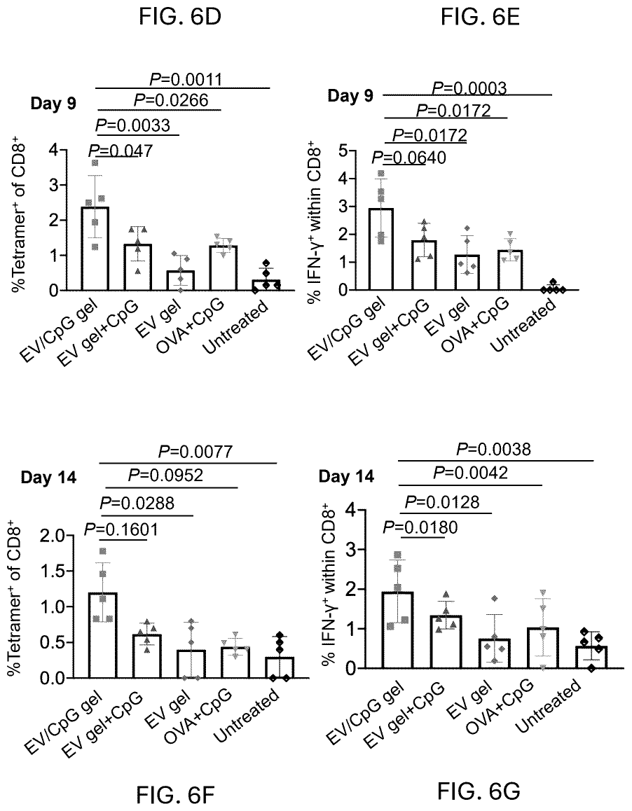

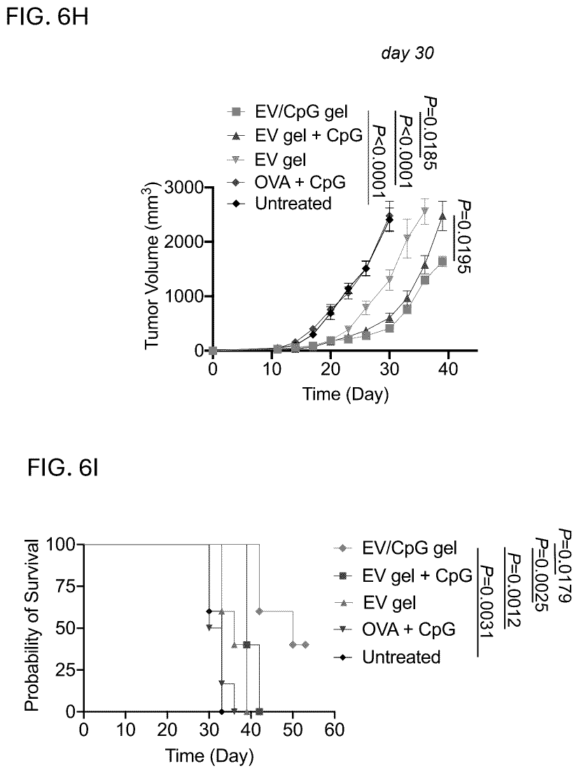

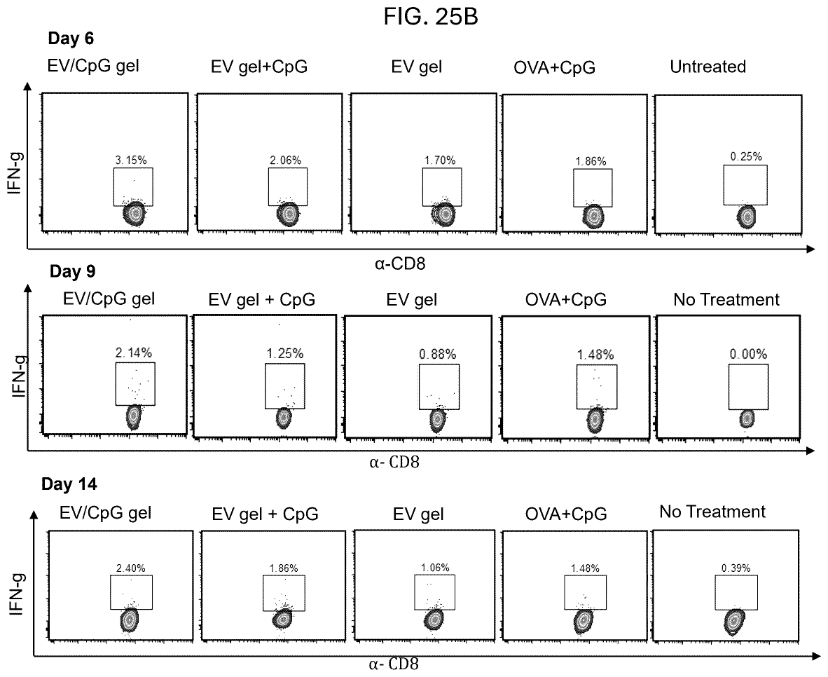

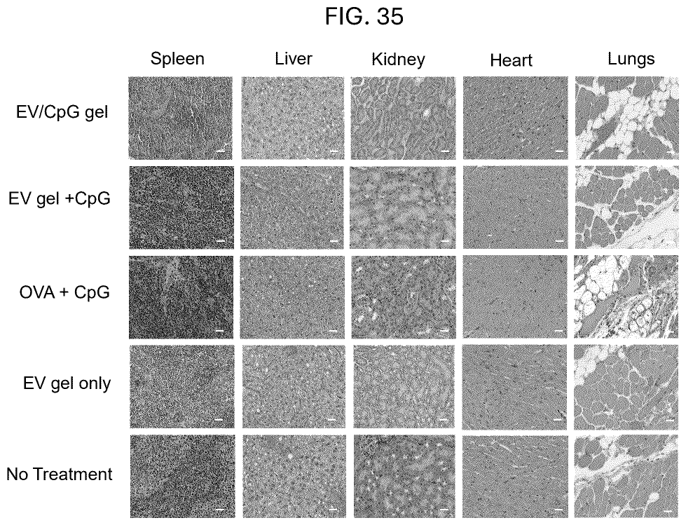

After demonstrating the excellent in vivo stability of EV gels and the presence of DCs and other immune cells at the gel site over time, we next studied whether EV gels can induce antigen-specific CTL response. E.G7-OVA derived EVs were used for this study. C57BL/6 mice were subcutaneously injected with CpG-conjugated EV gel, CpG-encapsulating EV gel, EV gel, the mixture of OVA (50 μg) and CpG (50 ng), and PBS, respectively on day 0 (FIG. 6A). Each injected EV gel contained 7×10⁸ EVs and 2.067 μg OVA. Peripheral blood mononuclear cells (PBMCs) were collected at different times for the analysis of SIINFEKL (SEQ ID NO: 1)-specific CD8⁺ T cells. On day 6, a significantly higher number of SIINFEKL (SEQ ID NO: 1)-specific CD8⁺ T cells was detected in mice treated with CpG-conjugated EV gels than in other groups (FIGS. 6B-6C). EV gels or the mixture of EV gel and CpG also induced a higher frequency of IFN-γ+CD8⁺ T cells, upon ex vivo SIINFEKL (SEQ ID NO: 1) restimulation, than the untreated group (FIG. 6C). On day 9, all treatment groups showed a higher number of SIINFEKL (SEQ ID NO: 1)-specific CD8⁺ T cells than the untreated group (FIGS. 6D-6E; FIGS. 25A-25B). Compared to EV gel alone or CpG-encapsulating EV gel, CpG-conjugated EV gels consistently induced a higher frequency of SIINFEKL (SEQ ID NO: 1)-specific CD8⁺ T cells in PBMCs on days 9 and 14 (FIGS. 6D-6G; FIGS. 25A-25B). In the subsequent prophylactic tumor study, all EV gel groups exhibited slower tumor growth than the untreated group (FIG. 6H). Compared to EV gel alone, CpG-conjugated EV gels resulted in slower tumor growth (FIG. 6H). EV gel treatment did not induce any noticeable toxicity to healthy tissues including spleen, liver, kidney, heart, and lungs (FIG. 35). These experiments demonstrated that CpG-conjugated EV gels can effectively modulate the DCs that arrive at the gel site and facilitate the processing and presentation of EV-encased antigens by DCs, thereby enhancing the antigen-specific CTL response.

### Example 7

### Antitumor Efficacy and Antibody Response of EV Gel Vaccines

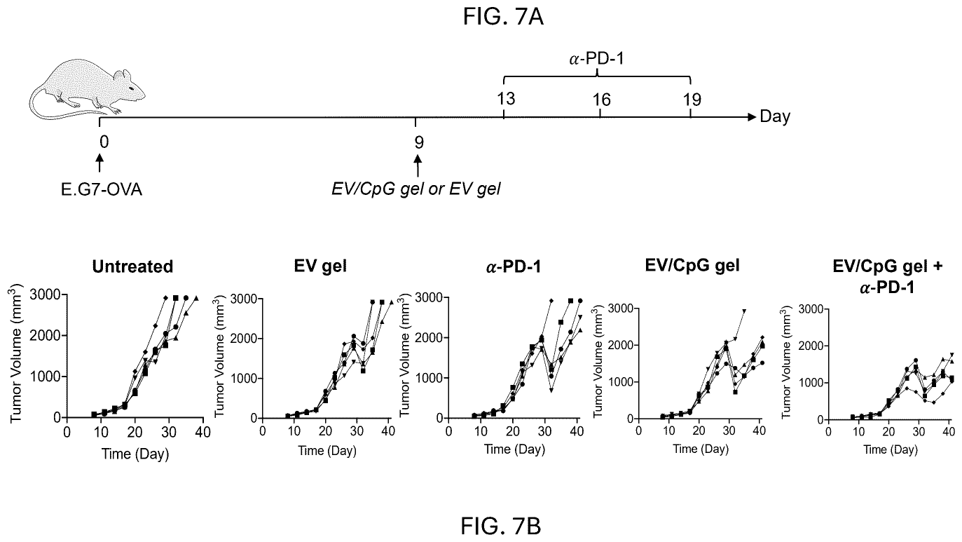

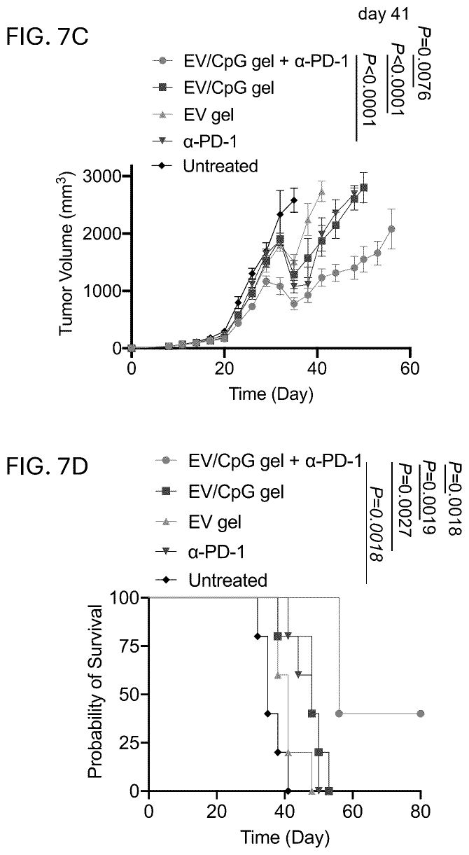

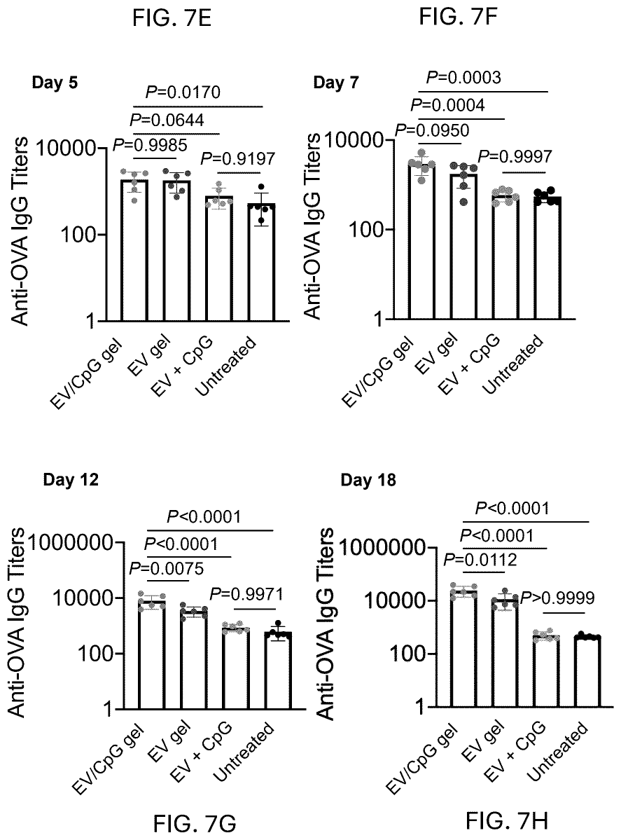

After demonstrating the enhanced CTL response and prophylactic antitumor efficacy of CpG-conjugated EV hydrogels, we next evaluated their therapeutic antitumor efficacy and potential synergy with anti-PD-1 checkpoint blockade. C57BL/6 mice were inoculated with E.G7-OVA tumors on day 0 and divided into 5 groups: the combination of CpG-conjugated EV gel and anti-PD-1, CpG-conjugated EV gel, EV gel, anti-PD-1, and PBS (FIG. 7A). EV gels were subcutaneously injected into the flank of mice on day 9, while anti-PD-1 was injected on days 13, 16, and 19, respectively (FIG. 7A). All treatment groups managed to slow down the growth of tumors in comparison with the untreated group (FIG. 7B). Compared to CpG-conjugated EV gel alone or anti-PD-1 alone, the combination of CpG-conjugated EV gel and anti-PD-1 led to an improved tumor control and prolonged animal survival (FIGS. 7C-7D). These experiments demonstrated the promise of EV gel-based cancer vaccines to induce enhanced CTL response and antitumor efficacy, and the synergistic effect between EV gel vaccines and checkpoint blockades. As a proof-of-concept demonstration, we also evaluated the promise of the EV gel to induce long-term humoral responses. C57BL/6 mice were injected with CpG-conjugated EV gels, EV gels, or the mixture of EVs and CpG on day 0, followed by the analysis of anti-OVA titers in the serum at different times. At all examined times (days 5, 7, 12, and 18), CpG-conjugated EV gels consistently generated higher anti-OVA titers than the bonus mixture of EV and CpG (FIGS. 7E-7H). Compared to EV gels, CpG-conjugated EV gels showed further increased anti-OVA titers from day 12 (FIGS. 7E-7H).

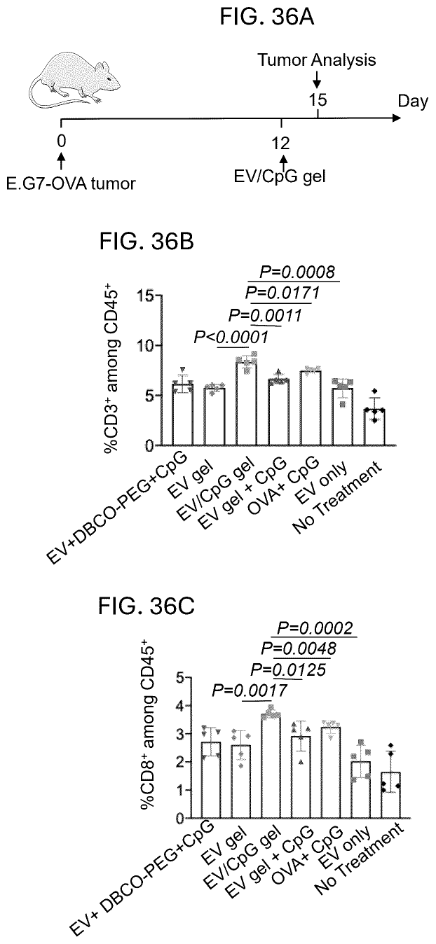

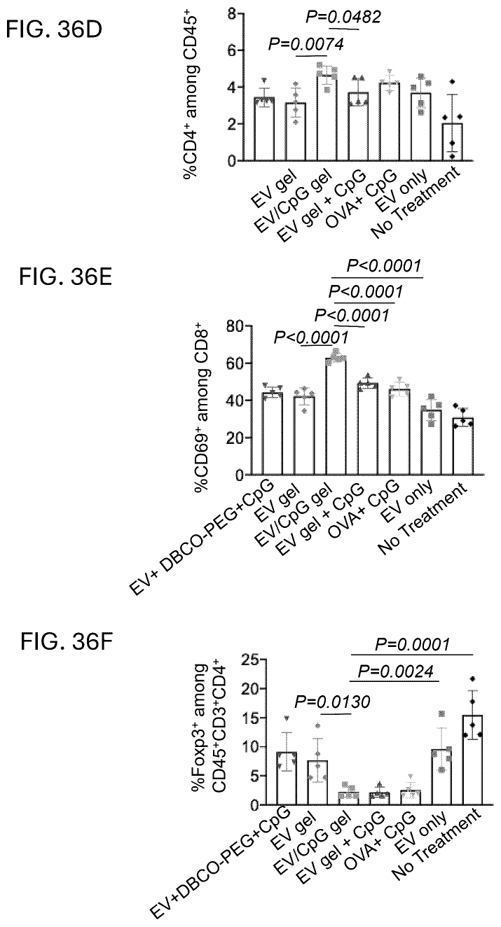

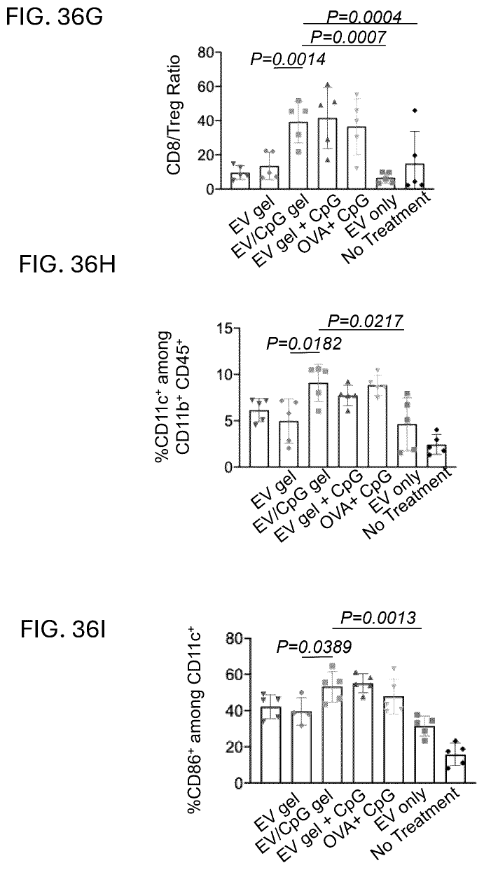

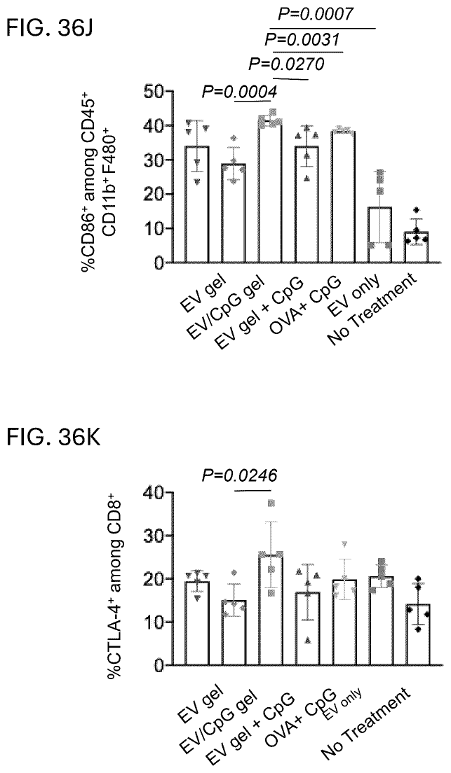

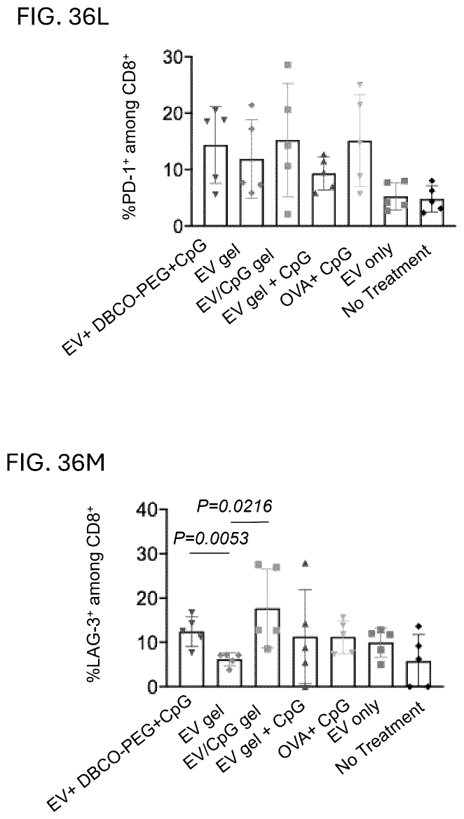

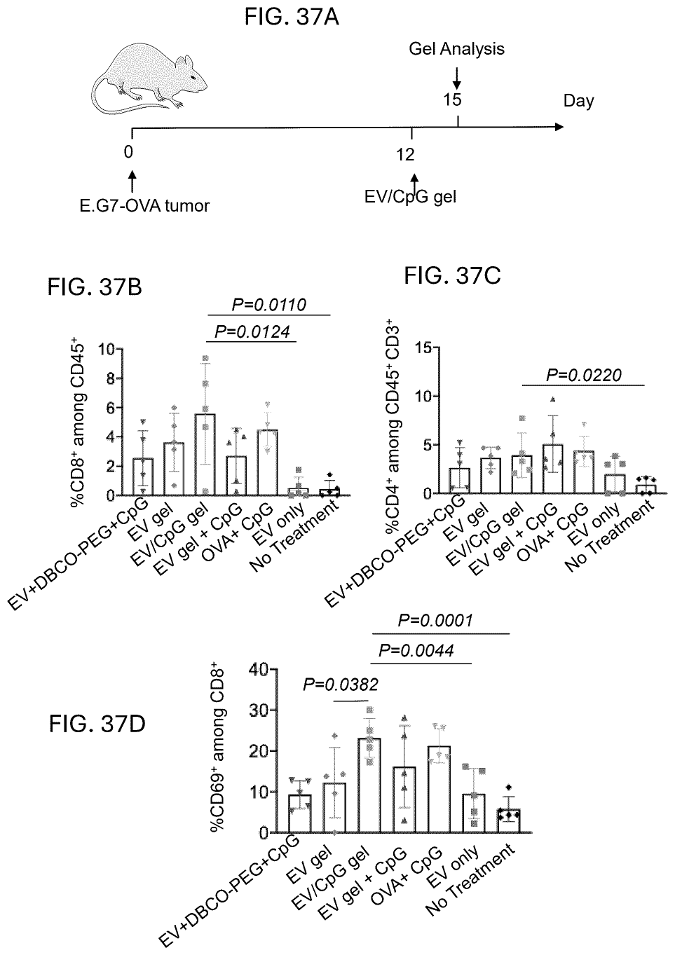

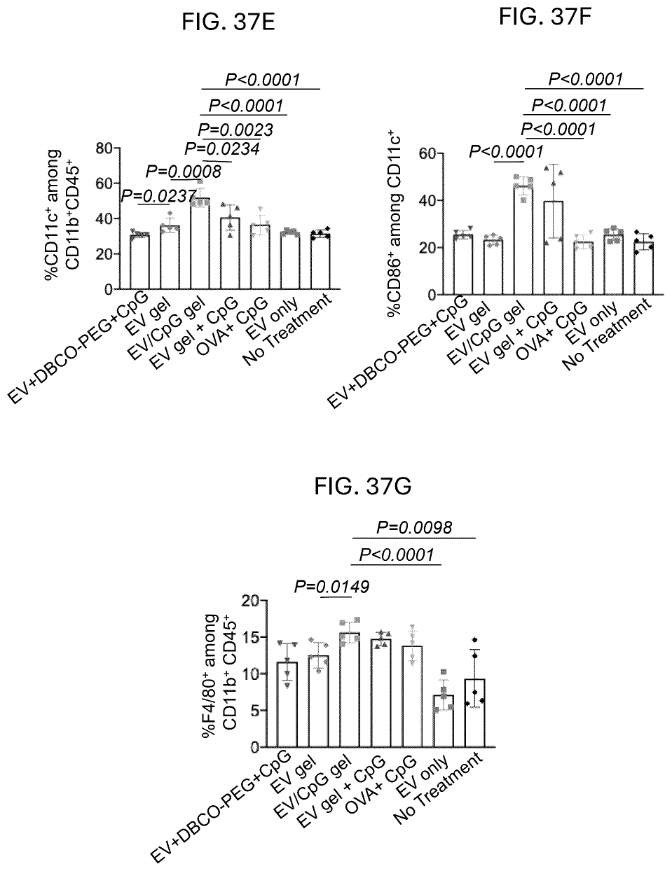

To understand the alteration of the tumor microenvironment as a result of EV gel treatment, mice bearing E.G7-OVA tumors were treated with CpG-conjugated EV gel, CpG-encapsulating EV gel, EV gel, the mixture of OVA (50 μg) and CpG (50 ng), EV alone, the mixture of EV, DBCO-PEG and CpG, and PBS, respectively. Tumors were harvested at 3 days post gel injection for immune cell analysis (FIG. 36A). Compared to EV gel or other groups, CpG-conjugated EV gel resulted in an increase in the number of intratumoral CD3⁺ T cells and CD8⁺ T cells (FIGS. 36B-36D), along with an upregulated expression of CD69 on the surface of intratumoral CD8⁺ T cells (FIG. 36E). CpG-conjugated EV gel also decreased the number of intratumoral regulatory T cells (Tregs) (FIG. 36F), increased the CD8⁺ T/Treg number ratio (FIG. 36G), increased the number of intratumoral DCs (FIG. 36H), and upregulated the expression of CD86 on the surface of intratumoral DCs (FIG. 36I), in comparison with EV gel, EV alone, the mixture of non-crosslinked EVs, DBCO-PEG and CpG. We also examined the expression of CTLA-4, PD-1, and LAG-3 exhaustion markers on the surface of CD8⁺ T cells in the tumor (FIGS. 36K-36M). Compared to EV gel or no treatment group, CpG-conjugated EV gels resulted in a slightly increased expression of CTLA-4 and LAG-3 on intratumoral CD8⁺ T cells (FIGS. 36K-36M). These experiments demonstrated the ability of CpG-conjugated EV gels to reprogram the immunosuppressive tumor microenvironment. We also analyzed the immune cells in the gel and surrounding skin tissues. Compared to EV alone or untreated group, CpG-conjugated EV gels increased the number of CD8⁺ T cells, CD4⁺ T cells, CD69⁺ CD8⁺ T cells, CD11c⁺ DCs, CD86⁺ DCs, and F4/80⁺ macrophages at the injection site (FIGS. 37A-37G). CpG-conjugated EV gels also resulted in a higher number of CD11c⁺ DCs, CD86⁺CD11c⁺ DCs, and F4/80⁺ macrophages than EV gels (FIGS. 37E-G).

It will be apparent that the precise details of the methods or compositions described may be varied or modified without departing from the spirit of the described aspects of the disclosure. We claim all such modifications and variations that fall within the scope and spirit of the claims below.

We claim:

1. A hydrogel comprising a plurality of extracellular vesicles covalently linked to a polymer, wherein the covalent link comprises a glycan moiety.

2. The hydrogel of claim 1, wherein the plurality of extracellular vesicles are covalently linked to the polymer by click chemistry.

3. The hydrogel of claim 2, wherein the click chemistry is azide-alkyne click chemistry, tetrazine-norbornene click chemistry, tetrazine-cyclooctene click chemistry, or maleimide-thiol click chemistry.

4. The hydrogel of claim 1, wherein the plurality of extracellular vesicles are from one or more tumor cells, stem cells, or pathogenic cells.

5. The hydrogel of claim 1, wherein the plurality of extracellular vesicles comprise exosomes.

6. The hydrogel of claim 1, wherein the polymer comprises polyethylene glycol, poly lactic-co-glycolic acid, or polycaprolactone.

7. The hydrogel of claim 1, wherein the polymer is modified with a linker.

8. The hydrogel of claim 7, wherein the linker comprises dibenzocyclooctyne.

9. The hydrogel of claim 1, further comprising an adjuvant.

10. A method of producing the hydrogel of claim 1, comprising:
providing a plurality of azido-labeled extracellular vesicles, wherein the azido-label is an azido-labeled glycan moiety; and
covalently coupling the plurality of azido-labeled extracellular vesicles to a polymer functionalized with a linker capable of covalently binding the azido-labeled extracellular vesicles.

11. The method of claim 10, wherein the azido-labeled glycan moiety is tetra-acetylated N-azidoacetyl-D-mannosamine (Ac₄ManNAz), tetra-acetylated N-azidoacetyl-D-galactosamine (Ac₄GalNAz), or tetra-acetylated N-azidoacetyl-D-glucosamine (Ac₄GlcNAz).

12. The method of claim 10, wherein the plurality of azido-labeled extracellular vesicles are produced by culturing a plurality of cells in the presence of an azido-labeled glycan moiety.

13. The method of claim 12, wherein the plurality of azido-labeled extracellular vesicles are obtained from cancer cells from a subject, stem cells, or pathogenic cells.

14. The method of claim 10, wherein the functionalized polymer comprises polyethylene glycol functionalized with dibenzocyclooctyne.

15. The method of claim 10, wherein covalently coupling the plurality of azido-labeled extracellular vesicles to the functionalized polymer comprises mixing the azido-labeled extracellular vesicles and the functionalized polymer and incubating at about 37° C. to form the hydrogel.

16. A method of treating a subject with a disease or disorder, comprising administering to the subject an effective amount of the hydrogel of claim 1, wherein the plurality of extracellular vesicles are derived from one or more tumor cells, stem cells or pathogenic cells.

17. The method of claim 16, wherein the subject has a tumor and the administering comprises injecting the hydrogel or a hydrogel precursor subcutaneously, at or near the tumor location, or intraperitoneally.

18. The method of claim 16, further comprising administering to the subject one or more immune checkpoint inhibitors.

19. The method of claim 18, wherein the one or more immune checkpoint inhibitors comprises one or more of an anti-PD-1 antibody, an anti-PD-L1 antibody, and/or an anti-CTLA-4 antibody.

20. The method of claim 16, wherein the subject has had a myocardial infarction and the extracellular vesicles are derived from stem cells, or the subject has a pathogenic infection and the extracellular vesicles are derived from pathogenic cells.
